(19) United States
(12) Patent Application Publication
(10) Pub. No.: US 2026/0367076 A1
(43) Pub. Date: Nov. 19, 2026

(54) MICROARRAYS

(71) Applicant: STANDARD BIOTOOLS INC., SOUTH SAN FRANCISCO, CA (US)

(72) Inventors: Jonathan M. BLACKBURN, RONDEBOSCH (ZA); Muneerah SMITH, RONDEBOSCH (ZA); Andrew NEL, RONDEBOSCH (ZA)

(73) Assignee: STANDARD BIOTOOLS INC., SOUTH SAN FRANCISCO, CA (US)

(21) Appl. No.: 19/142,620

(22) PCT Filed: Dec. 22, 2023
(86) PCT No.: PCT/US2023/085687

§ 371 Date: Jun. 23, 2025

(30) Foreign Application Priority Data
Dec. 23, 2022 (GB) 2219735.4

(51) Int. Cl. G01N 33/543; G01N 33/542; G01N 33/547

(52) U.S. Cl. CPC G01N 33/54353; G01N 33/542; G01N 33/547

(57) ABSTRACT

The present invention concerns microarrays. More particularly, this invention concerns low density microarrays, e.g. low density protein microarrays, which have low surface density of analyte within a discrete given area of the surface (e.g. a microarray spot). In addition, this invention relates to microarrays, e.g. protein microarrays, which have low surface coverage of analyte within a discrete given area of the surface (e.g. a microarray spot). The invention also concerns methods of manufacturing such microarrays.

### FIELD OF THE INVENTION

The present invention concerns microarrays. More particularly, this invention concerns low density microarrays, e.g. low density protein microarrays, which have low surface density of analyte within a discrete given area of the surface (e.g. a microarray spot). In addition, this invention relates to microarrays, e.g. protein microarrays, which have low surface coverage of analyte within a discrete given area of the surface (e.g. a microarray spot). The invention also concerns methods of manufacturing such microarrays.

### BACKGROUND OF THE INVENTION

Microarray technology enables the high throughput, parallel analysis of a number of different molecular interactions under uniform assay conditions. This makes microarrays a useful tool in many areas of bioscience research, such as the identification of novel disease-specific serological markers, the assessment of the activity of lead compounds in the drug discovery process against potential therapeutic targets and functional analysis of uncharacterised proteins.

Microarrays are typically ordered spatial arrangements of purified analytes, such as recombinant or native proteins. Multiple different analytes can be immobilised from nanolitre volumes in spatially-defined locations on a solid support, allowing high-throughput, miniaturised analytical (e.g. antibody binding) and functional (e.g. protein-protein, protein-DNA, or protein-small molecule interaction) assays to be performed on each immobilised analyte in parallel. Microarray technologies commonly used include both solid-state (planar or 2D) microarrays and bead-based suspension microarrays (Dunbar, S., et al. (2018). Solid and Suspension Microarrays for Detection and Identification of Infectious Diseases. In: Tang, YW., Stratton, C. (eds) Advanced Techniques in Diagnostic Microbiology. Springer, Cham. https://doi.org/10.1007/978-3-319-33900-9_20. The ideal microarray would produce highly sensitive and highly specific, quantitative data that accurately reflects the thermodynamics of true physiologically-relevant biomolecular interactions.

In respect of protein microarrays, in order to measure true physiologically-relevant biomolecular interactions in highly multiplexed protein array-based assays, each immobilised protein on the protein array needs to retain its physiologically-relevant folded structure and be presented on the surface in such a manner that true, specific biomolecular recognition events can occur. An ideal surface for protein microarray fabrication would thus capture and retain only folded proteins in a controlled orientation, whilst minimising non-specific interactions in array-based assays.

There are a number of common methods that are used for protein array fabrication, however, they all suffer from issues that reduce the effectiveness of the final protein array. Non-specific covalent coupling of proteins on to a surface results in immobilisation of proteins in random orientations, which can obscure functionally important regions of the protein from being accessed in the assay. Non-specific, non-covalent physisorption of proteins on to a surface, also results in immobilisation of proteins in random orientations and typically leads to protein unfolding and loss of activity on the surface. Encapsulation in a hydrogel can preserve protein conformation and function, but restricts access to macromolecular interactors, and affinity capture on to a surface offers no control over protein orientation.

Additionally, these methods for protein array fabrication all typically result in high densities of immobilised protein in each location on the resultant protein array, which can drive non-specific interactions, including non-specific aggregation-driven interactions, that have no physiological relevance. Furthermore, the immobilised protein molecules are typically bound in a fixed orientation on the surface, potentially occluding true binding sites.

By contrast, in a natural cellular environment, most soluble proteins are found at low concentrations and most membrane-bound proteins are found at low densities, allowing true, specific biomolecular interactions to occur and strongly thermodynamically disfavouring non-specific interactions. Moreover, in order for true physiologically-relevant biomolecular interactions to occur, the specific binding sites on interacting proteins need to be physically accessible and to be able to find each other in 3D diffusional space, which typically requires rotational and conformational freedom to be retained, especially for membrane-associated proteins.

Typically, protein arrays known in the art are blocked using a proteinaceous reagent, most commonly bovine serum albumin, casein or milk powder, in order to reduce non-specific binding of macromolecules to the surface, but these blocking reagents themselves show significant non-specific binding to other macromolecules.

Thus, protein arrays known in the art are not always well suited to the measurement of true physiologically-relevant biomolecular interactions in highly multiplexed assays, since it is typically difficult to distinguish true, specific binding on the array surface from a background of non-specific interactions. They suffer from high non-specific background binding, as well as poor accessibility of specific binding sites on fixed-orientation immobilised proteins. They also have no control over the density of immobilisation and do not enable localised rotational or conformational freedom on the surface.

Thus, there is a need in the art for an improved protein microarray that can make multiplexed, quantitative, physiologically-relevant measurements of biomolecular interactions across multiple different proteins in parallel.

### SUMMARY OF THE INVENTION

The present invention provides according to a first aspect, a microarray comprising a surface to which are bound a plurality of reactive groups, wherein an analyte is immobilised on said surface via direct binding to one or more of the reactive groups or via indirect binding via a linking moiety to one or more of the reactive groups, and wherein the surface density of said analyte within at least one discrete given area of said surface is less than about 20%. As part of this first aspect, the present invention also provides a microarray comprising a surface to which are bound a plurality of reactive groups, wherein an analyte is immobilised on said surface via direct binding to one or more of the reactive groups or via indirect binding via a linking moiety to one or more of the reactive groups, and wherein the surface coverage of said analyte within at least one discrete given area of said surface is less than about 20%.

According to a second aspect, the present invention provides a method of manufacturing a microarray comprising the steps of: (i) providing a surface to which are bound a plurality of reactive groups, wherein only approximately 20% or less of said reactive groups are able to react at any one time; (ii) optionally contacting said reactive groups with a linking moiety under conditions whereby said linking moiety reacts with one or more reactive groups which is able to react, resulting in the binding of the linking moiety to said surface; and (iii) depositing a sample of an analyte on the surface within at least one discrete given area of the surface such that the analyte is immobilised on said surface via direct binding to one or more reactive groups which is able to react, or via indirect binding via said linking moiety to a reactive group; wherein the surface coverage of the bound analyte within the at least one discrete given area of said surface is accordingly less than about 20%.

According to a third aspect, the present invention provides a method of reducing the density of analyte bound to the surface of a microarray comprising the steps of (i) providing a surface to which are bound a plurality of reactive groups; (ii) causing or allowing a portion of said reactive groups to be deactivated or inaccessible such that approximately 20% or less of said reactive groups are able to react at any one time; (iii) optionally contacting said reactive groups with a linking moiety under conditions whereby one or more reactive groups retaining reactivity are able to react with the linking moiety resulting in the binding of the linking moiety to said surface; and (iv) depositing a sample of an analyte on the surface within at least one discrete given area of the surface such that the analyte is immobilised on said surface via direct binding to one or more reactive groups, or via indirect binding via said linking moiety to one or more reactive groups; wherein the surface coverage of the bound analyte within the at least one discrete given area of said surface is accordingly less than about 20%.

According to a fourth aspect, the present invention provides a method of:
i) increasing the analyte signal to background noise ratio of a microarray;
ii) increasing the rotational and conformational freedom of an analyte immobilised on a microarray; and/or
iii) increasing the proportion of physiologically-relevant interactions between analytes immobilised on a microarray and test molecules applied to said microarray;
   wherein said method comprises the step of providing a microarray comprising a surface to which are bound a plurality of reactive groups, wherein the analyte is immobilised on said surface via direct binding to one or more of the reactive groups or via indirect binding via a linking moiety to one or more of the reactive groups, and wherein the surface coverage of the analyte immobilised within at least one discrete given area of said surface is less than about 20%.

According to a fifth aspect, the present invention provides a use of a surface of a microarray as a low density surface on which the surface coverage (or surface density) of analyte immobilised within at least one discrete given area of said surface is less than about 20%, wherein the analyte is optionally immobilised on said microarray via a linking moiety.

According to a sixth aspect, the present invention provides a use of a surface for the manufacture of a microarray comprising immobilised analyte, wherein the surface coverage (or surface density) of analyte immobilised within at least one discrete given area of said surface is less than about 20% and wherein the analyte is optionally immobilised on said microarray via a linking moiety.

According to a seventh aspect, the present invention provides a use of reactive groups of a surface suitable for forming a microarray for reducing the density of analyte immobilised on said surface, wherein a proportion of said reactive groups are unable to react with said analyte such that the surface coverage (or surface density) of analyte immobilised on at least one discrete given area of said surface is less than about 20%, and wherein said analyte is optionally immobilised on said surface via a linking moiety.

According to an eighth aspect, the present invention provides a use of reactive groups of a surface suitable for forming a microarray for the manufacture of a low density protein microarray, wherein a proportion of said reactive groups on said surface are unable to react with an analyte such that the surface coverage (or surface density) of analyte immobilised on at least one discrete given area of said surface is less than about 20%, and wherein said analyte is optionally immobilised on said surface via a linking moiety.

In a ninth aspect, as disclosed herein, the present invention is directed to the use of a microarray as defined in the first aspect for:
i) the identification of interactions between said analyte and test molecules applied to said analyte;
ii) the determination of an antibody profile of a subject;
iii) identifying a biomolecule which specifically binds to said immobilised analyte;
iv) the identification of an antibody which specifically binds said immobilised analyte and which is suitable for the diagnosis or treatment of a disease; or
v) identifying a biomolecule which specifically binds said immobilised analyte and which is capable of treating a disease mediated by said immobilised analyte.

Further, in a tenth aspect of the invention as disclosed herein, there is provided a method of manufacturing a microarray comprising the steps of: (i) providing a surface to which are bound a plurality of reactive groups; (ii) contacting said reactive groups with a linking moiety comprising a biotin-binding molecule under conditions whereby said linking moiety reacts with one or more reactive groups which is able to react, resulting in the binding of the linking moiety to said surface; (iii) depositing a sample of a biotinylated analyte such that the analyte is immobilised on said surface via indirect binding via said linking moiety to a reactive group; and thereafter (iv) applying a solution of biotin to the surface of the microarray.

In an eleventh aspect of the invention, there is provided a method of increasing the analyte signal to background noise ratio of a microarray comprising the steps of: (i) providing a surface to which are bound a plurality of reactive groups, wherein only approximately 20% or less of said reactive groups are able to react at any one time; (ii) contacting said reactive groups with a linking moiety, which is a biotin-binding moiety, under conditions whereby said linking moiety reacts with one or more reactive groups which is able to react, resulting in the binding of the linking moiety to said surface; (iii) depositing a sample of a biotinylated analyte on the surface within at least one discrete given area of the surface such that the analyte is immobilised on said surface via indirect binding via said linking moiety to a reactive group; wherein the surface coverage (surface density) of the bound analyte within a given area of said surface is accordingly less than about 20% and thereafter (iv) applying a solution of biotin to the surface of the microarray.

In a twelfth aspect of the invention, there is provided a microarray comprising a surface to which are bound a plurality of reactive groups, wherein an analyte is immobilised on said surface via direct binding to one or more of the reactive groups or via indirect binding via a linking moiety to one or more of the reactive groups, and wherein the number density of said analyte (i.e. the number of immobilised analyte molecules) within at least one discrete given area of said surface is less than about 300 per micrometre squared.

In a thirteenth aspect of the invention, there is provided a method of manufacturing a microarray comprising the steps of: (i) providing a surface to which are bound a plurality of reactive groups; (ii) contacting said reactive groups with a solution of a linking moiety wherein the concentration of the linking moiety in said solution is less than 1 mg/ml and whereby said linking moiety reacts with one or more reactive groups which is able to react, resulting in the binding of the linking moiety to said surface.

In a fourteenth aspect of the invention, the invention provides for a microarray obtainable by any of the methods of the tenth or thirteenth aspects of the invention.

It will be appreciated that features described in relation to one aspect of the present invention may be incorporated into other aspects of the present invention. For example, the methods and uses of the invention may incorporate any of the features described with reference to the microarrays of the invention and vice versa.

### DESCRIPTION OF THE DRAWINGS

FIGS. 1A and 1B show quality control checks to determine the reproducibility of fabricated slides. Both figures show Cy5 biotin-labelled bovine serum albumin (Cy5 BBSA) fluorescence to demonstrate slide coating homogeneity (FIG. 1A) and spot uniformity (FIG. 1B).

FIG. 2 shows confirmation of His-tagged CYP450 binding using an anti-6×His probe.

FIG. 3 is a flow chart showing an experimental plan for comparing biotin versus BSA treatment of replica protein arrays post-array fabrication.

FIG. 4A shows the layout on the microarray of BCCP-tagged full length and truncated SARS-COV-2 Nucleocapsid protein antigens. FIG. 4B shows the array image, scanned at 532 and 635 nm, for the biotin-blocked array. FIG. 4C shows the array image, scanned at 532 and 635 nm, for the BSA-blocked array.

FIGS. 5A and 5B show a comparison of the foreground signal intensity (at 635 nm) of the printed spots on the biotin-blocked array (FIG. 5A) and on the BSA-blocked array (FIG. 5B).

FIG. 6 shows a comparison of the signal intensity (at 635 nm) of surrounding background areas adjacent to printed spots for a biotin-blocked array (FIG. 6A) and for a BSA-blocked array (FIG. 6B).

FIG. 7 shows a scanning atomic force microscopy (AFM) topographical image of a 1 μm by 1 μm area within one CYP450 spot on the protein array.

FIGS. 8A and 8B both show schematic representations of embodiments of the invention in which a test molecule (5) interacts with an analyte (4) immobilised on a surface (1), e.g. of a microarray. In FIG. 8A, the analyte (4) is bound to a reactive group (2) on the surface via a linking moiety (3), whereas in FIG. 8B, the analyte (4) is bound directly to a reactive group (2) on the surface. The analyte (4) can be for example a recombinant or native protein, a BCCP-tagged protein, a biotinylated protein, a nucleic acid, a sugar, a bacterium, or another type of molecule. The linking moiety can be for example streptavidin. The reactive group (2) can be for example an NHS-activated PEG polymer. In both cases, the reactive group (2) is bound to a surface (1), which can be for example a glass slide, a plate well, a bead surface, or other surface. The block lines represent linkages between the individual elements (surface (1), reactive group (2), linking moiety (3) if present and analyte (4)). Such a linkage may be any suitable linkage, e.g., a covalent bond or a non-covalent interaction, e.g., the interaction between streptavidin and biotin. The dashed line represents an interaction between the analyte and a test molecule (5), which may be, for example, a protein-ligand interaction, or an antibody-antigen interaction.

FIG. 9A shows fluorescence images of Nexterion H slides coated with streptavidin solutions at concentrations from 0.05 to 2 mg/ml and incubated with Cy3-biotin-BSA.

FIG. 9B shows fluorescence images of Nexterion H slides coated with streptavidin solutions at concentrations from 0.05 to 2 mg/ml in the presence of 50 mM glycine as a competitor and incubated with Cy3-biotin-BSA.

FIG. 9C shows fluorescence images of aged Nexterion H slides derivatised with streptavidin solutions at concentrations from 0.03 to 2 mg/ml and incubated with Cy3-biotin-BSA.

FIG. 9D shows fluorescence images of Nexterion H slides derivatised with streptavidin solution at a concentration of 1 mg/ml at pH 9 or pH 4.5 and incubated with Cy3-biotin-BSA, as well as negative controls (without streptavidin) including slide coating buffer, slide coating buffer with 50 mM glycine or an empty gasket well, also incubated with Cy3-biotin-BSA.

FIG. 9E shows binding curves for Nexterion H slides derivatised with streptavidin alone, in the presence of 50 mM glycine, or on aged H slides, then incubated with Cy3-biotin-BSA.

FIG. 9F shows a bar graph of average fluorescence intensity for Nexterion H slides derivatised with streptavidin at pH 8.5, 9 or 4.5 and incubated with Cy3-biotin-BSA.

FIG. 9G shows a bar graph of average fluorescence intensity of negative control Nexterion H slides with coating buffer only (no glycine), slide coating buffer with 50 mM glycine (Glycine) or an empty well, then incubated with Cy3-biotin-BSA.

FIG. 10 shows streptavidin binding on Nexterion H slides derivatised with streptavidin solutions at concentrations of 0.125-8 mg/ml streptavidin and incubated with Cy3-biotin-BSA with or without a subsequent block step with 50 mM biotin.

FIG. 11A shows an SEM image of a Nexterion H slide derivatised with 8 mg/ml streptavidin and incubated with Cy3-biotin-BSA.

FIG. 11B shows an SEM image of a Nexterion H slide derivatised with 0.25 mg/ml streptavidin and incubated with Cy3-biotin-BSA.

FIG. 11C shows an SEM image of a Nexterion H slide incubated with slide coating buffer only (negative control) and incubated with Cy3-biotin-BSA.

FIG. 12 shows the microarray layout where SARS-COV-2 proteins, including the full-length S ectodomain trimer and the C-terminal domain of the N protein, were printed in triplicate on Nexterion H slides derivatised under various conditions.

FIG. 13A shows an anti-c-Myc assay for the detection of SARS-COV-2 S and CTD proteins detected on Nexterion H slides derivatised with 0.03-2 mg/ml streptavidin.

FIG. 13B shows an anti-c-Myc assay for the detection of SARS-COV-2 S and CTD proteins detected on Nexterion H slides derivatised with 0.03-2 mg/ml streptavidin in the presence of 50 mM glycine.

FIG. 13C shows an anti-c-Myc assay for the detection of SARS-COV-2 S and CTD proteins detected on aged Nexterion H slides derivatised with 0.03-2 mg/ml streptavidin.

FIG. 13D shows an anti-c-Myc assay for the detection of SARS-COV-2 S and CTD proteins detected on H slides derivatised with streptavidin at pH 9 and pH 4.5, as well as negative controls including slide coating buffer (SCB) only, slide coating buffer with 50 mM glycine (SCB+50 mM glycine) or an empty well.

FIG. 14A shows microarray images of IgG detection against SARS-COV-2 S proteins printed on Nexterion H slides derivatised with 0.03-2 mg/ml streptavidin.

FIG. 14B shows microarray images of IgG detection against SARS-COV-2 S proteins printed on Nexterion H slides derivatised with 0.03-2 mg/ml streptavidin in the presence of 50 mM glycine.

FIG. 14C shows microarray images of IgG detection against SARS-COV-2 S proteins printed on aged Nexterion H slides derivatised with 0.03-2 mg/ml streptavidin.

FIG. 14D shows microarray images of IgG detection against SARS-COV-2 S proteins printed on Nexterion H slides derivatised with streptavidin at pH 9 and pH 4.5, as well as negative controls including slide coating buffer (SCB) only, slide coating buffer with 50 mM glycine (SCB+50 mM glycine) or an empty well.

FIG. 15A shows an IgG assay for SARS-COV-2 S proteins detected on Nexterion H slides derivatised with 0.03-2 mg/ml streptavidin.

FIG. 15B shows an IgG assay for SARS-COV-2 S proteins detected on Nexterion H slides derivatised with 0.03-2 mg/ml streptavidin in the presence of 50 mM glycine.

FIG. 15C shows an IgG assay for SARS-COV-2 S proteins detected on aged Nexterion H slides derivatised with 0.03-2 mg/ml streptavidin.

FIG. 15D shows an IgG assay for SARS-COV-2 S proteins detected on Nexterion H slides derivatised with streptavidin at pH 9 and pH 4.5.

### DETAILED DESCRIPTION

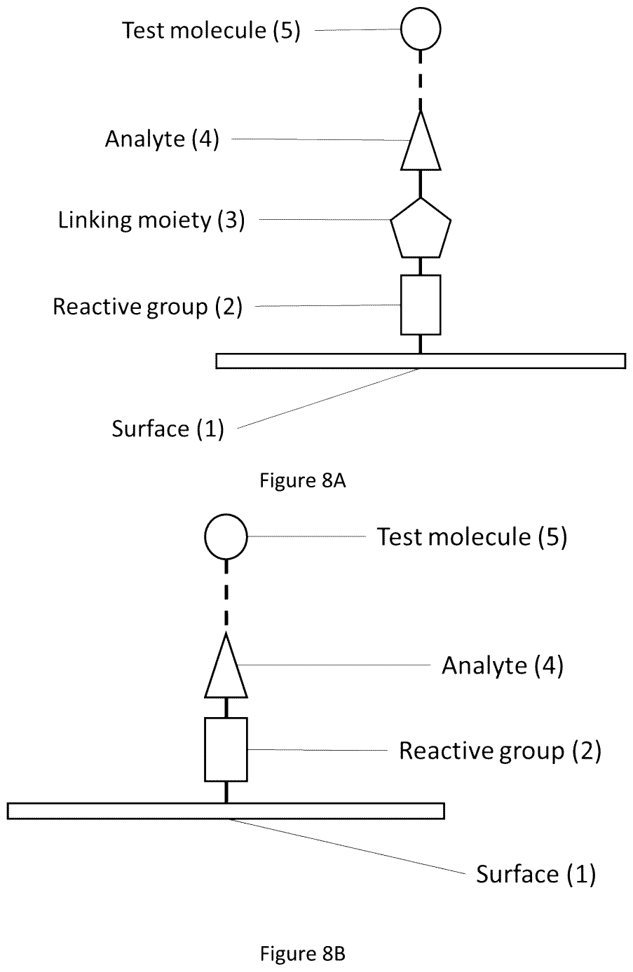

The present invention is directed to an improved microarray, in particular a microarray on which analytes are immobilised at low density on the surface of the microarray. For a complete understanding of the terminology used in the context of the present invention (e.g. “analyte”, “linking moiety”, “reactive group”, “surface” and “test molecule”), reference is made to FIGS. 8A and 8B and the accompanying description thereof. As can be seen from those figures, an “analyte” in the context of the present invention is a moiety which is immobilised on a microarray and for which it is desired to analyse the properties thereof. Such analysis may, for example, take the form of determining the interactions of the analyte with a “test molecule” which is applied to the immobilised analyte. In certain embodiments of the invention, the analyte is immobilised on a surface of the microarray via direct binding to a “reactive group” on the surface. In other embodiments, the analyte is immobilised on a surface of the microarray via indirect binding via a “linking moiety” to a reactive group on the surface.

In more detail, the present invention is based on the surprising observation that immobilising analytes at low density of the surface of the microarray leads to improved properties—for example, when the immobilised analytes are well separated from each other on the surface of the array, they are less likely to aggregate and they retain localised rotational and conformational freedom. This enables the immobilised analytes to behave as if they are in a cellular environment, resulting in substantially improved biomolecular interaction specificity (with greater physiological relevance), lower limits of detection, and significantly reduced non-specific binding in array-based assays, since such non-specific binding is thermodynamically disfavoured by the arrangement of the present invention. In particular, the low density arrays of the present invention permit the immobilised analytes to be more physically accessible, with reduced steric hindrance and/or occlusion of relevant binding sites. Thus, the risk of within-spot, inter-analyte steric hindrance that may prevent proper interaction of analytes with binding partners is reduced. Such immobilised analytes are thus better able to find their (physiologically relevant) interacting molecules (e.g. binding partners) in 3D diffusional space. Such interactions include, for example, protein-protein, protein-ligand, protein-nucleic acid, and protein-small molecule interactions.

Furthermore, where the analyte of the present invention is a protein or polypeptide, the surfaces of the microarrays of the present invention favour the capture of correctly-folded proteins/polypeptides in a controlled orientation, thus again favouring physiologically relevant interactions (with applied test molecules), whilst minimising non-specific interactions, such as non-specific aggregation-driven interactions. Accordingly, the microarrays of the present invention allow the immobilised analytes to behave more as though they are in a natural cellular environment, where, for example, most soluble proteins are found at low concentrations and most membrane-bound proteins are found at low densities. Immobilisation in a controlled (fixed) orientation is not only on one array, but across all arrays, which enhances reproducibility of interactions with a given analyte.

In protein arrays known in the art, such arrays are typically blocked using a proteinaceous reagent (most commonly bovine serum albumin, casein or milk powder) in order to reduce non-specific binding of macromolecules to the surface. However, such blocking reagents themselves show significant non-specific binding to other macromolecules, thus resulting in interactions which are not physiologically relevant. The present invention does not require blocking with a proteinaceous reagent, thus avoiding the resulting disadvantages.

A further advantage associated with the present invention (where the bound analyte is a protein or polypeptide) is the use of a protein folding marker to report on the folded state of individual recombinant proteins. Thus, such a folding marker can be used to immobilise only folded and biologically functional proteins (at low density) on to the surface of the array. One of the major advantages of this is that interacting/binding partners actually require the 3D conformational sites generated by folding for specific analyte recognition and binding, allowing in vivo-like interaction specificity; such sites are lost if the protein is not correctly folded.

Due the above advantages, the interactions of the analytes immobilised on the arrays of present invention exhibit high-specificity, low background, and disfavoured non-specific interactions. Accordingly, the arrays of the present invention generate relevant analyte interactions with applied test molecules which have high signal to noise ratio. This provides for accurate quantitative measurements with low limits of detection. Furthermore, due to the particular technology used within the context of the arrays of the present invention to immobilise the analytes on the surface of the array, the present invention allows the immobilisation and purification of the desired analyte to be combined in a single step, as described in greater detail elsewhere herein.

Finally, the methods of manufacturing a microarray as described herein allow control of the density at which proteins are immobilised on the surface of the microarray.

In a first aspect, the present invention is directed to a microarray comprising a surface to which are bound a plurality of reactive groups, wherein an analyte is immobilised on said surface via direct binding to one or more of the reactive groups or via indirect binding via a linking moiety to one or more of the reactive groups, and wherein the surface density of said analyte within at least one discrete given area of said surface is less than about 20%, e.g, wherein the surface density of said analyte within an analyte spot on said microarray is less than about 20%.

As part of this first aspect, the present invention is also directed to a microarray comprising a surface to which are bound a plurality of reactive groups, wherein an analyte is immobilised on said surface via direct binding to one or more of the reactive groups or via indirect binding via a linking moiety to one or more of the reactive groups, and wherein the surface coverage of said analyte within at least one discrete given area of said surface is less than about 20% of said given area. The microarray may be referred to as a low density microarray or a microarray having low surface coverage of immobilised analyte, e.g, wherein the surface coverage of said analyte within at least one analyte spot on said microarray is less than about 20% of the area of said analyte spot.

In an embodiment of this first aspect, the present invention is directed to a low density microarray comprising a surface to which are bound a plurality of reactive groups, wherein an analyte is immobilised on said surface via direct binding to one or more of the reactive groups or via indirect binding via a linking moiety to one or more of the reactive groups, and wherein the surface coverage of said analyte within at least one discrete given area of said surface is less than about 20% of said given area.

In an embodiment of this first aspect, the surface density of said analyte within at least one discrete given area of said surface is less than about 18%, e.g. less than about 16%, less than about 14%, less than about 12%, less than about 10%, less than about 8%, less than about 6%, less than about 4% or less than about 2%.

In an embodiment of this first aspect, the surface coverage of or by said analyte within at least one discrete given area of said surface is less than about 18%, e.g. less than about 16%, less than about 14%, less than about 12%, less than about 10%, less than about 8%, less than about 6%, less than about 4% or less than about 2% of said given area.

In an embodiment of this first aspect, the surface density of said analyte within at least one discrete given area of said surface is at least about 0.05%, preferably at least about 0.5%, more preferably at least about 1%. For example, in an embodiment of this first aspect, the surface density of said analyte within at least one given area of said surface is in the range of about 0.05% to about 20%, about 0.5% to about 20%, about 1% to about 20%, about 0.05% to about 15%, about 0.05% to about 12%, about 0.05% to about 10%, about 0.5% to about 10% or about 1% to about 10%.

In an embodiment of this first aspect, the surface coverage of or by said analyte within at least one discrete given area of said surface is at least about 0.05%, preferably at least about 0.5%, more preferably at least about 1% of said given area. For example, in an embodiment of this first aspect, the surface coverage of said analyte within at least one discrete given area of said surface is in the range of about 0.05% to about 20%, about 0.5% to about 20%, about 1% to about 20%, about 0.05% to about 15%, about 0.05% to about 12%, about 0.05% to about 10%, about 0.5% to about 10% or about 1% to about 10% of said given area.

In the context of the present invention, the “surface coverage” of an analyte within a discrete given area means the percentage proportion of a particular discrete area on the microarray (e.g. the proportion of the area of an individual analyte “spot” on the surface of the microarray) that is covered by bound analyte molecules. Surface coverage can be measured, for example, by microscopy, for example by atomic force microscopy (AFM), or for example by scanning electron microscopy (SEM). If the surface coverage of an analyte within a given area is referred to as being less than 10%, this means that analyte molecules cover less than 10% of the given area (e.g. a spot) of the array.

In an embodiment of this first aspect, the surface coverage of said analyte within substantially all, or all the discrete given areas on the surface (i.e. all the individual analyte “spots” on the surface of the microarray) is less than about 20%, e.g. is less than about 18%, e.g. less than about 16%, less than about 14%, less than about 12%, less than about 10%, less than about 8%, less than about 6%, less than about 4% or less than about 2%. For example, in an embodiment of this first aspect, the surface coverage of said analyte within substantially all, or all the discrete given areas on the surface (i.e. all the individual analyte “spots” on the surface of the microarray) is in the range of 0.05% to 20%, 0.5% to 20%, 1% to 20%, 0.05% to 15%, 0.05% to 12%, 0.05% to 10%, 0.5% to 10% or 1% to 10% of said given area.

As will be apparent, in an embodiment, a “discrete given area” as referred to herein is an analyte “spot” on the surface of the microarray, i.e. an area where analyte is printed onto the microarray. Outside of said discrete given areas, analyte may not be immobilised on the surface of the microarray.

Thought of in an alternative way, a reference to the surface coverage of an analyte within a given area (as referred to herein) means the percentage of the reactive groups within a given area of a surface of a microarray, which are actually bound (either directly or indirectly via a linking moiety) to analyte. Taking a simple example, which is purely for the purposes of illustration, if a given area of a surface of a microarray is characterised as containing a 10×10 grid of reactive groups (i.e. 100 reactive groups in total in the given area), then a surface coverage of less than 10% would correlate to fewer than 10 of those 100 reactive groups being bound (either directly or indirectly via a linking moiety) to analyte. It should be noted that, in the context of the present invention, a reference to a low surface coverage array does not refer to the density (number) of spots which are on the array (or how close such spots are to each other), but rather the percentage of the area of each individual microarray spot on which analyte molecules are immobilised. In this context, it should be noted that references to “high density arrays” may be a term in the art, but that term conventionally refers to an array in which a high density (number) of spots are printed on the array, i.e. a high number of spots are printed close to one another on the array. This is thus a different concept from how the terms surface density or surface coverage are used in the context of the present invention.

The surface density of analyte in the context of the invention can alternatively be thought of as the number of analyte molecules that are immobilised on a microarray, per unit area. An alternative definition of low surface density can therefore be defined as fewer than a given number of immobilised analyte molecules per unit area, optionally when analysed by atomic force microscopy (AFM), or optionally when analysed by scanning electron microscopy (SEM).

In an embodiment of this first aspect, the number of immobilised analyte molecules is fewer than about 300 per micrometre squared, e.g. fewer than about 275 per micrometre squared, fewer than about 250 per micrometre squared, fewer than about 225 per micrometre squared, fewer than about 200 per micrometre squared, fewer than about 175 per micrometre squared, fewer than about 150 per micrometre squared, fewer than about 125 per micrometre squared, fewer than about 100 per micrometre squared, fewer than about 75 per micrometre squared, fewer than about 50 per micrometre squared, fewer than about 25 per micrometre squared, fewer than about 20 per micrometre squared, fewer than about 15 per micrometre squared, fewer than about 10 per micrometre squared or fewer than about 5 per micrometre squared, optionally when analysed by AFM, or optionally when analysed by SEM.

Alternatively, the surface density or surface coverage of analyte in the context of the invention can be thought of as the number of reactive groups, e.g. NHS-activated PEG polymers, that are bound to a linking moiety, e.g. a streptavidin tetramer, per unit area. Tagged (e.g. biotinylated) analyte will only bind specifically to the linking moiety (e.g. streptavidin). Therefore, it is the distribution of linking moieties (such as streptavidin) bound to a reactive group (e.g. an NHS-activated PEG polymer) across the surface of the slide that ultimately controls the density or coverage of analyte bound to the slide. In the case of reactive groups which are PEG polymers (e.g. NHS-activated PEG polymers), although the proportion of PEG polymers that are NHS-activated is very high, virtually 100%, the present inventors have determined that the proportion of NHS-activated polymers that are able and available to bind to a linking moiety such as streptavidin can be made to be surprisingly low. This means that the surface density or surface coverage of bound linking moiety (e.g. streptavidin) can be low, consequently resulting in the low surface density or low surface coverage of bound analyte. Therefore, alternatively, a low surface density can be defined as fewer than a given number of linking moieties bound per unit area of the surface of a microarray, when analysed, for example, by AFM, or, for example by SEM. In an embodiment of the first aspect of the invention, the number of linking moieties (e.g. streptavidin) bound to the surface of the microarray is fewer than about 300 per micrometre squared, e.g. fewer than about 275 per micrometre squared, fewer than about 250 per micrometre squared, fewer than about 225 per micrometre squared, fewer than about 200 per micrometre squared, fewer than about 175 per micrometre squared, fewer than about 150 per micrometre squared, fewer than about 125 per micrometre squared, fewer than about 100 per micrometre squared, fewer than about 75 per micrometre squared, fewer than about 50 per micrometre squared, fewer than about 25 per micrometre squared, fewer than about 20 per micrometre squared, fewer than about 15 per micrometre squared, fewer than about 10 per micrometre squared or fewer than about 5 per micrometre squared, optionally when analysed by AFM, or optionally when analysed by SEM. In a similar way, a low surface coverage can be defined as less than a certain percentage of a given area of the surface being covered by linking moieties when analysed, for example, by AFM, or, for example by SEM. In an embodiment of the first aspect of the invention, the surface coverage of or by the linking moiety within a given area of said surface is less than 20%, e.g. less than 18%, less than 16%, less than 14%, less than 12%, less than 10%, less than 8%, less than 6%, less than 4% or less than 2%, optionally when analysed by AFM. In an embodiment of this first aspect, the surface coverage of or by the linking moiety within a given area of said surface is at least 0.05%, preferably at least 0.5%, more preferably at least 1%. For example, in an embodiment of this first aspect, the surface coverage of or by the linking moiety within a given area of said surface is in the range of 0.05% to 20%, 0.5% to 20%, 1% to 20%, 0.05% to 15%, 0.05% to 12%, 0.05% to 10%, 0.5% to 10% or 1% to 10%.

For the avoidance of doubt, references herein to a “given area” refer to a sub-area of the surface of the microarray (e.g. a “spot”) and not the entire area of the surface of the microarray. An individual “given area” (e.g. an analyte “spot” or “dot”) may have a diameter (if circular) of approximately 200 μm and thus an area of about 0.03 mm². In an embodiment of this first aspect, a “given area”, as referred to herein, has a diameter (if circular) in the range of 1 μm to 500 μm. In an embodiment of this first aspect, a “given area”, as referred to herein, has a surface area in the range of from 0.80 μm² to 0.2 mm². As will be apparent, a microarray will normally have multiple “given areas” (e.g. multiple “spots”) on the surface. Such “given areas” may be printed at high density on the surface (i.e. close together), but within those given areas, the analytes are immobilised at low density.

Additionally, it should be noted that, in all the aspects and embodiments of the present invention, within the given area, (e.g. a microarray “spot”), analyte molecules (and linking moieties if present) are advantageously evenly distributed across the surface of that given area. In other words, the arrays of the present invention and the methods of preparing them do not result in a given area which contains sub-areas where analyte molecules (and linking moieties if present) are immobilised at high density (“clumping”) interspersed with sub-areas where surface-bound analyte is essentially absent, which would result in an overall average low surface density.

The microarrays of the present invention may therefore comprise one or, more typically, a plurality of spots, each spot constituting a “given area” as defined herein. Around or intermediate the one or more spots, the microarray may comprise one or more interspot regions (which may be contiguous) which comprise no or substantially no analyte. Those skilled in the art will understand that the interspot regions may comprise reactive groups and optionally linking moieties. After depositing analyte molecules onto the microarray as one or more spots, unbound reactive groups and/or linking moieties on the microarray may be blocked as disclosed herein.

In the context of all of the aspects of the present invention, a reference to a “surface” of a microarray should be interpreted not only to refer to a flat slide (i.e. a solid-state, planar or 2D microarray, e.g. made of glass), but also other suitable surfaces such as a bead (as is found in a suspension or 3D microarray), or the well of a plate (e.g. a 96 well plate). Accordingly, the term “microarray” should be interpreted to include both planar, solid-state microarrays and suspension microarrays. In an embodiment of each of the aspects of the invention, the microarray is a planar microarray (also known as a solid-state or 2D microarray). In another embodiment of each of the aspects of the invention the microarray is a suspension microarray, for example a bead-based suspension microarray (also known as a 3D microarray). Such suspension arrays of beads (which may also be referred to as microspheres) are well-known in the art (Nolan, J. P. and Sklar, L. A., (2002) Trends in Biotechnology, vol. 20 (1), p. 9-12, https://doi.org/10.1016/S0167-7799 (01) 01844-3).

In the context of all of the aspects and embodiments of the present invention, a reference to surface density may alternatively be read as a reference to surface coverage and vice versa as appropriate.

In an embodiment of this first aspect, the analyte is immobilised on said surface via indirect binding to one or more of the reactive groups via a linking moiety.

In an embodiment of this first aspect, the analyte comprises a tag which permits immobilisation of said analyte on said surface.

In an embodiment of this first aspect, the analyte comprises one or more of a polypeptide, a nucleic acid, a lipid and a carbohydrate.

For the purposes of this invention, a protein is considered to be a polypeptide chain of 50 or more amino acids, a peptide is considered to be a polypeptide chain of fewer than or equal to 50 amino acids, but greater than 20 amino acids, and an oligopeptide is considered to be a polypeptide chain of at least two, and fewer than or equal to 20 amino acids. Accordingly, as defined herein, oligopeptides, peptides and proteins are collectively referred to as polypeptides.

In an embodiment of this first aspect, the analyte is a polypeptide (i.e. an oligopeptide, a peptide or a protein), or a nucleic acid.

In an embodiment of this first aspect, the analyte is a polypeptide (i.e. an oligopeptide, a peptide or a protein), for example a glycoprotein.

In an embodiment of this first aspect, the analyte is biotinylated, for example a biotinylated polypeptide.

5-[(3aS,4S,6aR)-2-Oxohexahydro-1H-thieno[3,4-d]imidazol-4-yl]pentanoic acid, otherwise known as biotin, is a small chemical compound widely found in living organisms and is involved in a number of metabolic processes. The protein streptavidin has extremely high affinity for biotin. With a dissociation constant (K_{d}) of approximately ~10⁻¹⁴ M, the binding of biotin to streptavidin is one of the strongest non-covalent interactions known in nature. For the purposes of the present invention, the term biotin encompasses biotin itself and derivatives thereof which retain the functionality of biotin, i.e. which have a binding affinity for streptavidin of less than 10⁻⁹ M, for example less than 10⁻¹² M.

In an embodiment of this first aspect, the biotinylated analyte, for example a biotinylated polypeptide, is a chemically biotinylated analyte or an enzymatically biotinylated analyte. In an embodiment of this first aspect, the biotinylated analyte, for example a biotinylated polypeptide, is an enzymatically biotinylated polypeptide.

In an embodiment of this first aspect, the analyte is a polypeptide, preferably a biotinylated polypeptide, and said tag which permits immobilisation of said polypeptide on said surface is fused to the N- or the C-terminus of the polypeptide.

In an embodiment of this first aspect, the polypeptide is correctly folded. For the purposes of this invention, a “correctly folded” polypeptide means that the polypeptide chain has arranged itself into the appropriate secondary or tertiary structure and is functionally active.

Polypeptides or other analytes contemplated herein can be biotinylated at available primary amine groups. In polypeptides, such primary amine groups may be those present in lysine side-chains or at the N-terminal or C-terminal residues. In these cases, the biotin molecule becomes covalently bound to the polypeptide after a dehydration reaction between the carboxyl group of the biotin and the amine group of the polypeptide.

Biotinylation can be carried out chemically or enzymatically. When done enzymatically, the reaction is catalysed by a biotin ligase, which recognises the biotinylation motif methionine-lysine-methionine in the polypeptide of interest. Such a biotinylation motif can be found, for example, in a biotin carboxyl carrier protein (BCCP) domain. Biotin ligases preferentially biotinylate the lysine in this motif. A polypeptide of interest can be expressed with a BCCP domain fused to it (e.g. to the N- or C-terminus), in, for example, insect cells (such as Spodoptera frugiperda cells) or in E. coli. Such methods of biotinylation can thus be used to control the site of biotinylation on the protein. Methods of in vitro biotinylation can also be employed using E. coli biotin ligase (BirA), as described in Schatz, P. J. (1993), Bio/Technology (Nature Publishing Company), 11(10), 1138-1143. https://doi.org/10.1038/nbt1093-1138. Such methods are useful for certain BCCP-tagged proteins (e.g. secreted, membrane-bound) which are not biotinylatable within insect cells due to maturation within the endoplasmic reticulum (ER) which does not contain the biotinylation machinery present in the cytosol.

Chemical methods provide greater flexibility in the type of biotinylation needed than enzymatic approaches and can be performed both in vitro and in vivo. Furthermore, chemical methods do not require the co-expression of bacterial biotin ligase and the modification of the polypeptide of interest to carry a biotin acceptor peptide. Chemical biotinylation reagents carry a reactive moiety which is able to cross-link the biotin to a reactive moiety in the polypeptide of interest (e.g. a primary amine, a sulfhydryl, a carboxyl or a carbonyl). Non-selective biotinylation reagents are also available which can be used to label macromolecules which have no available primary amines, sulfhydryls, carboxyls or carbonyls. A so-called spacer arm may be present between the reactive moiety and the biotin molecule itself. Amines are the most commonly targeted functional groups for biotinylation because of the abundance of lysine side chain ε-amines and N-terminal α-amines. N-hydroxysuccinimide (NHS) esters readily form stable bonds with primary amines, and the reactive group is easily incorporated and stabilized into a variety of useful, ready-to-use biotinylation reagents. NHS-esters can be modified to be water-soluble by sulfonating the N-hydroxysuccinimide ring to form sulfo-NHS esters. Tetrafluorophenyl (TFP) esters comprise a commonly used amine-reactive group that has similar reactivity with primary amines but is more hydrophobic than NHS. Sulfhydryl groups, which are found in exposed cysteine residues, are the second most-common targets for biotinylation. Examples of reactive moieties in Sulfhydryl-reactive biotinylation reagents include maleimide, iodoacetyl and pyridyl disulphide groups. Carboxyl groups are found on the carboxy-terminal ends of proteins and on asparate and glutamate side chains. Biotinylation reagents that target carboxyl groups require a zero-length crosslinker such as EDC (a carbodiimide) to conjugate to primary amines on the biotinylation reagents. So, while the amines on carboxyl-reactive biotinylation reagents are not reactive per se, they are the site of conjugation to target proteins. Besides amines, biotinylation reagents with hydrazide moieties can also be used with EDC to react with carboxyl groups. Although carbonyls do not readily exist in proteins, carbohydrate residues on glycoproteins can be modified to aldehydes to be labeled with hydrazide or alkoxyamine derivative biotinylation reagents. Aldehydes on these glycoproteins are generated by the oxidation of carbohydrate sialic acids using sodium periodate. The aldehydes are then reacted specifically with a hydrazide or alkoxyamine at pH 4-6 to form a stable linkage. Nonselective, photoreactivatable biotinylation reagents are available to label target proteins without available amines, sulfhydryls, carboxyls and carbohydrates. Most photoreactive biotinylation reagents are based on aryl azides, which become activated by UV light (>350 nm) and initiates an addition reaction to insert into C—H and N—H sites. Subsequent ring expansion drives the reaction towards binding to nucleophiles, such as primary amines. Usually, photoactivatable reagents are chosen when primary amines and other functional groups are lacking or when the initiation of conjugation must be timed to a particular point in an incubation period (i.e., by exposure to UV light).

In an embodiment of this first aspect, the tag which permits immobilisation of the analyte on the surface of the microarray comprises a biotin carboxyl carrier protein (BCCP) motif, an Avi-tag (SEQ ID NO: 1), a SNAP-tag®, a SpyTag or SpyCatcher protein. An Avi-tag is a 15 residue peptide (sequence—GLNDIFEAQKIEWHE, defined herein as SEQ ID NO: 1) that mimics the biotin acceptor function of the much larger BCCP domain normally recognised by biotin ligase. The advantage of this is that the Avi-tag is much smaller than the BCCP domain and so can be used as the site of biotinylation on a recombinant protein where potential steric conflicts need to be minimised. The SNAP-tag® is a self-labeling protein tag which is commercially available from New England Biolabs, Inc. in various expression vectors. SNAP-tag® is a 182-residue polypeptide (19.4 kDa) that can be fused to an analyte of interest, e.g. a polypeptide, and further specifically and covalently tagged with a suitable ligand, for example, biotin. An analyte of the present invention may thus be fused with a SNAP-tag® and then labelled with biotin. The peptide SpyTag (13 amino acids) spontaneously reacts with the protein SpyCatcher (12.3 kDa) to form an intermolecular isopeptide bond between the pair (PNAS, 109 (12) E690-E697 https://doi.org/10.1073/pnas.1115485109). The SpyTag or the SpyCatcher protein can be attached to an analyte of interest, for example, DNA sequence encoding SpyTag or SpyCatcher can be recombinantly introduced into the DNA sequence encoding a polypeptide analyte of interest, forming a fusion protein, with the cognate pair being bound to the surface of the microarray. The analyte fusion proteins can then be covalently bound to the microarray via reaction through the SpyTag/SpyCatcher system.

In an embodiment of this first aspect, the tag which permits immobilisation of the analyte on the surface of the microarray comprises a BCCP motif or an Avi-tag (SEQ ID NO: 1). In an embodiment of this first aspect, the tag which permits immobilisation of the analyte on the surface of the microarray comprises a biotin carboxyl carrier protein (BCCP) motif.

In an embodiment of this first aspect, the BCCP motif has at least 80% sequence identity, preferably at least 90% sequence identity, more preferably 100% sequence identity to SEQ ID NO. 2. In an embodiment of this first aspect, the BCCP motif is preferably derived from E. coli and corresponds to residues 74-156 of the AccB protein, i.e. AAAEISGHIV RSPMVGTFYR TPSPDAKAFI EVGOKVNVGD TLCIVEAMKM MNQIEADKSG TVKAILVESG QPVEFDEPLV VIE (defined herein as SEQ ID NO. 2). This BCCP motif is cross-recognised by eukaryotic biotin ligases, enabling it to be biotinylated efficiently in yeast, insect and mammalian cells without the need to co-express the E. coli biotin ligase. Usefully, the N- and C-termini of the BCCP domain are physically separated from the site of biotinylation by 50 Å, which is ideal for presenting the recombinant protein away from the surface, thus minimising any deleterious effects due to immobilisation. In an embodiment of this first aspect, insect cells are used to express the recombinant BCCP-tagged protein. In an embodiment of this first aspect, the insect cells are Spodoptera frugiperda cells. Insect cell expression systems allow complex eukaryotic post-translational modifications (which preserve protein function and epitope detection), whilst being compatible with mild lysis conditions. Mammalian systems are also compatible with expression and biotinylation of BCCP tagged recombinant proteins.

In an embodiment of this first aspect, the BCCP motif is correctly folded. For the purposes of this invention, a “correctly folded” BCCP motif refers to a motif that is an appropriate substrate for a biotin ligase enzyme, e.g. E. coli biotin ligase. Usefully, it has been demonstrated that biotinylation of the BCCP motif in a fusion protein is a reliable marker of the folded state of the fusion partner, with only correctly folded recombinant fusion proteins becoming biotinylated by biotin ligase enzymes.

In an embodiment of this first aspect, the biotin is bound to: (i) a biotin attachment domain within said BCCP motif or (ii) said Avi-tag.

In an embodiment of this first aspect, the biotin is attached to said biotin attachment domain within said BCCP motif or said Avi-tag via enzymatic biotinylation, preferably via a biotin ligase, e.g. E. coli biotin ligase.

In an embodiment of this first aspect, the linking moiety is a biotin-binding molecule and said analyte is biotinylated.

In an embodiment of this first aspect, the linking moiety which is a biotin-binding molecule is a protein.

In an embodiment of this first aspect, the linking moiety which is a biotin-binding molecule is selected from the group consisting of (i) an anti-biotin antibody; (ii) avidin; (iii) neutravidin; (iv) streptavidin, or (iii) a fragment, variant or analogue of streptavidin, avidin, neutravidin or said anti-biotin antibody which retains biotin-binding capability. In the context of this embodiment of this first aspect of the invention, a fragment, variant or analogue of streptavidin, avidin, neutravidin or said anti-biotin antibody which retains biotin-binding capability has a binding affinity for biotin of less than 10⁻⁹ M, for example less than 10⁻¹² M, for example less than 10⁻¹³ M.

Avidin is a tetrameric biotin-binding protein that is found naturally in egg whites. Each monomeric unit of the protein is capable of binding biotin and this binding interaction between avidin and biotin is one of the strongest known non-covalent interactions, with a dissociation constant of approximately 10⁻¹⁵ M.

Neutravidin is a deglycosylated homolog of avidin. This reduces non-specific binding of lectin and decreases the pI of the molecule to near-neutral, thus reducing non-specific binding of charged molecules such as nucleic acids.

Streptavidin is another tetrameric biotin-binding protein that was originally isolated from the bacterium Streptomyces avidinii. Streptavidin has a relatively low primary sequence identity to avidin, but the two molecules share almost identical secondary and tertiary structures. Consequently, streptavidin also has a similarly high binding interaction with biotin to avidin. However, streptavidin has a superior binding affinity for biotin-conjugate molecules than avidin, and like neutravidin, also has a neutral charge.

In an embodiment of this first aspect, the biotin-binding molecule comprises a sequence having at least 80% sequence identity, at least 90% sequence identity or 100% sequence identity to SEQ ID NO. 3, which is the amino acid sequence of the core streptavidin molecule (residues 37-159) of Streptomyces avidinii, i.e. AEAG ITGTWYNQLG STFIVTAGAD GALTGTYESA VGNAESRYVL TGRYDSAPAT DGSGTALGWT VAWKNNYRNA HSATTWSGQY VGGAEARINT QWLLTSGTTE ANAWKSTLVG HDTFTKVKP. In an embodiment of this first aspect, the biotin-binding molecule comprises a sequence having at least 80% sequence identity, at least 90% sequence identity or 100% sequence identity to SEQ ID NO. 4, which is the amino acid sequence of the full length streptavidin molecule (residues 1-183) of Streptomyces avidinii, i.e. MRKIVVAAIAVSLTTVSITASASADPSKDSKAQVSAAEAGITGTWYNQLGSTFIVTA GADGALTGTYESAVGNAESRYVLTGRYDSAPATDGSGTALGWTVAWKNNYRNAHSAT TWSGQYVGGAEARINTQWLLTSGTTEANAWKSTLVGHDTFTKVKPSAASIDAAKKAG VNNGNPLDA. In an embodiment of this first aspect, the biotin-binding molecule, e.g. streptavidin, is a tetramer (i.e. it is in tetrameric form). In an embodiment of this first aspect, the biotin-binding molecule, e.g. streptavidin, is a tetramer (i.e. it is in tetrameric form) and is not aggregated. In an embodiment of this first aspect, the biotin-binding molecule, e.g. streptavidin, is a tetramer (i.e. it is in tetrameric form), is not aggregated and is bound to 1, 2, 3 or 4 biotinylated analyte molecules. Where the streptavidin tetramer is bound to 3 or fewer biotinylated analyte molecules, the remaining biotin binding sites in the tetramer may be bound to free biotin.

In an embodiment of this first aspect, the microarray comprises multiple binding sites for biotin (i.e. via the biotin-binding molecules, such as streptavidin), wherein at least a portion of said biotin binding sites are not bound to biotinylated analyte, and wherein said biotin binding sites which are not bound to biotinylated analyte are bound to free biotin. This may be achieved by treating the surface of the array with a solution containing free biotin after immobilisation of a biotinylated analyte. This results in a significant reduction in the background, non-specific binding of applied test molecules to the surface (by a factor of ~60) (applied test molecules are molecules which are applied to the array to determine whether they can bind to the immobilised analyte). This is unexpected and very useful, since it gives a large increase in signal to noise. In an embodiment of this first aspect, substantially all of the biotin binding sites on the microarray which are not bound to biotinylated analyte, are bound to free biotin, for example at least 70%, at least 80%, at least 90%, at least 95%, at least 98% or at least 99% of said biotin binding sites which are not bound to biotinylated analyte, are bound to free biotin. By free biotin it is meant biotin that is not bound to analyte, for example the bare biotin molecule. In an embodiment of this first aspect, said biotin-binding molecule is streptavidin. Streptavidin comprises four binding sites for biotin, since it is a tetramer. Accordingly, in an embodiment of this first aspect (when said biotin-binding molecule is streptavidin), in substantially all of the streptavidin tetramers present on the surface of the array, all four biotin binding sites in said streptavidin tetramers are bound to either biotinylated analyte or free biotin. In this context, by “substantially all” it is meant that in at least 70%, in at least 80%, in at least 90%, in at least 95%, in at least 98% or in at least 99% of the streptavidin tetramers present on the surface of the array, all four biotin binding sites in said streptavidin molecules are bound to either biotinylated analyte or free biotin. For example, one binding site may be bound to biotinylated analyte and the remaining three binding sites may be bound to free biotin. In another example, two binding sites may be bound to biotinylated analyte and the remaining two binding sites may be bound to free biotin.

The very high affinity (Kd~10⁻¹⁴ to 10⁻¹⁵ M) and specificity of the streptavidin-biotin interaction circumvents the need to perform laborious pre-purification of each expressed, biotinylated recombinant protein prior to array fabrication. Array fabrication thus becomes simpler as the crude lysate containing the expressed recombinant-biotinylated protein is printed onto streptavidin-coated slides which have a low non-specific protein-binding capacity. The printed microarray can then be washed removing non-biotinylated proteins from the array surface.

The high affinity of the streptavidin-biotin interactions allows for the rapid saturation of available biotin-binding sites on the surface. This means that a crude normalization of protein loading can be achieved without pre-adjusting the concentrations of the crude lysates to compensate for differences in the individual expression levels of different recombinant proteins.

In an embodiment of this first aspect, the surface density or surface coverage is calculated using microscopy.

In an embodiment of this first aspect, the surface density or surface coverage is calculated using a microscopy technique selected from the group consisting of atomic force microscopy (AFM), electron microscopy (EM), e.g. scanning electron microscopy (SEM) and super-resolution microscopy. In an embodiment of this first aspect, the surface density or surface coverage is calculated using AFM. In an embodiment of this first aspect, the surface density or surface coverage is calculated using AFM when used in contact mode with an AFM pin of thickness 2 μm. In an embodiment of this first aspect, the surface density or surface coverage is calculated using AFM when used in contact mode with an AFM pin of thickness 2 μm and tip scans are performed using either 50 μm² or 1 μm² areas. In an embodiment of this first aspect, the surface density or surface coverage is calculated using SEM. In an embodiment of this first aspect, the surface density or surface coverage is calculated using SEM, wherein the sample to be analysed is coated with a layer of carbon (e.g. of 5 nm thickness, e.g. using a Quorum Q150VEplus combined carbon/splutter coater) and then scanned, e.g. using a Tescan MIRA3 electron microscope, under appropriate magnification (e.g. 100,000×) and using an appropriate voltage (e.g. 10 kV).

Electron microscopy refers to microscopy techniques that use electrons as the radiation. Transmission electron microscopy (TEM) is very similar in configuration to traditional light microscopy, using lenses to focus a radiation beam onto a slide prepared with a slice of a sample, albeit in this case the radiation used is electrons rather than visible light. TEM is advantageous over traditional light microscopy because the wavelength of an electron is multiple orders of magnitude smaller than that of visible light, resulting in a much greater degree of resolution, typically less than one nanometre, compared to the theoretical limit of 200 nm imposed by light microscopes. One major difference in the preparation of samples for TEM is that they must be stained with heavy metals in order to create contrast in the image. Scanning electron microscopy (SEM) is related, but instead of passing an electron beam through a slice of a sample, the beam is reflected off the surface of the sample and so can be used to create a 3-dimensional image of a sample. The resolution achievable in SEM is typically around 50 nm. A major consideration of both EM methods is that the system requires imaging to be performed in a vacuum, in order not to disrupt the path of the electrons.

Atomic force microscopy (AFM) is a type of scanning microscopy, see Microsc. Res. Tech. 2017; 80:75-84 (DOI 10.1002/jemt.22776). In most typical configurations, a cantilever is scanned over the surface of the sample, whilst a laser is shone onto the cantilever. As the cantilever moves across the sample's surface, a small tip on the cantilever contacts the surface and causes the cantilever to bend in response to the interaction with the sample. The change in laser light that is measured reflecting off the cantilever can be used to construct an image of the sample. A distinct advantage of this method over other high-resolution microscopy methods is that AFM does not require the use of any lenses, nor a vacuum, nor any sample staining methods to be effective, with a typical image resolution of less than one nanometre.

Super-resolution microscopy refers to optical microscopy techniques such as structured-illumination microscopy (SIM), stochastic optical reconstruction microscopy (STORM) and Photoactivated localization microscopy (PALM) that allow images to be recorded with a resolution higher than the diffraction limit of traditional light microscopy. Super-resolution microscopes typically have a resolution of 10-100 nm. An advantage of this method is that it is compatible with use of multiple fluorophore-labelled reagents such as antibodies, enabling simultaneous, multiplexed identification of specific proteins components in a sample (e.g. in a cell or on a surface).

In an embodiment of this first aspect, the reactive groups on said surface of the microarray are selected from the group consisting of carboxylic acid groups, activated carboxylic acid groups, amine groups, imidoester groups, maleimide groups, haloacetyl groups, pyridyl dithiol groups, azide groups, hydrazide groups, alkoxyamine groups, thiol groups, aryl azide groups and diazirine groups.

In an embodiment of this first aspect, the reactive groups on said surface are selected from the group consisting of carboxylic acid groups, activated carboxylic acid groups, amine groups and maleimide groups.

In an embodiment of this first aspect, the reactive groups on said surface are activated groups, for example 1-ethyl-3-(3-carboxylic acid dimethylaminopropyl) carbodiimide (EDC) activated or N-hydroxysuccinimide (NHS) activated carboxylic acid groups.

In an embodiment of this first aspect, a proportion of the reactive groups on the surface are unable to react, for example, about 1%, about 2%, about 5%, about 10%, about 15%, about 20%, about 25%, about 50%, about 75% or about 90% of the reactive groups are unable to react. Reactive groups on the surface which are unable to react may be physically inaccessible or may have become deactivated with time (e.g. the reactive group, such an NHS ester may have spontaneously hydrolysed). Alternatively, the reactive groups on the surface may be actively deactivated, e.g. via a specific chemical treatment. In an embodiment of this first aspect, the microarray is a 2D solid state slide (e.g. a Nexterion H slide) comprising reactive groups which is beyond the manufacturer's recommended use by date. In an embodiment of this first aspect, the microarray is a 2D solid state slide (e.g. a Nexterion H slide) comprising reactive groups which has been stored, e.g. at −20 °C, for at least 3 months, at least 6 months, at least 1 year, at least 18 months or at least 2 years before use. Such slides have a lower proportion of reactive groups (e.g. NHS esters) which are able to react due to spontaneous hydrolysis of said reactive groups over time.

In an embodiment of this first aspect, the reactive groups are bound to said surface of the microarray via a hydrophilic organic polymer.

In an embodiment of this first aspect, the hydrophilic organic polymer is selected from the group consisting of polyacrylamide, polyurethane, polyethyleneimine and polyethylene glycol, preferably polyethylene glycol.

In an embodiment of this first aspect, the hydrophilic organic polymer is a polyethylene glycol having an average molecular weight in the range 500 to 20000, for example 500 to 15000, 500 to 10000, 500 to 7500, 500 to 5000 or 1000 to 5000. Specific PEGs include PEG1000, PEG2000, PEG3000, PEG3500 and PEG5000. Since PEG itself blocks non-specific adsorption sites on the glass slide, this circumvents the requirement for additional proteinaceous surface-blocking agents which themselves may alter surface-bound analyte behaviour or give rise to non-specific binding.

In an embodiment of this first aspect, the immobilised analytes or the linking moieties on said surface are spaced at least 10, at least 20, at least 50, at least 75, at least 100, at least 180, at least 200, at least 250 or at least 500 nm apart from each other, e.g. as measured by AFM or SEM. In an embodiment of this first aspect, the immobilised analytes or the linking moieties on said surface are spaced at least 50 or at least 100 nm or at least 180 nm apart from each other, e.g. as measured by AFM or SEM. In an embodiment of this first aspect, the immobilised analytes or the linking moieties on said surface are spaced at least 50 nm, at least 100 nm, at least 150 nm or at least 200 nm apart from each other, for example between 50 and 500 nm, between 50 and 250 nm, between 50 and 200 nm, between 50 and 150 nm or between 50 and 100 nm apart, e.g. as measured by AFM or SEM. When making measurements by AFM according to this embodiment, the optimal empirically-determined recommendations and parameters for factors such as calibration, oscillation amplitude, cantilever/probe tip characteristics, cantilever stability, and image processing pipeline should be followed.

In a second aspect, the present invention is directed to a method of manufacturing a microarray comprising the steps of: (i) providing a surface to which are bound a plurality of reactive groups, wherein only approximately 20% or less of said reactive groups are able to react at any one time; (ii) optionally contacting said reactive groups with a linking moiety under conditions whereby said linking moiety reacts with one or more reactive groups which is able to react, resulting in the binding of the linking moiety to said surface; and (iii) depositing a sample of an analyte on the surface within at least one discrete given area of the surface such that the analyte is immobilised on said surface via direct binding to one or more reactive groups which is able to react, or via indirect binding via said linking moiety to a reactive group; wherein the surface density of the bound analyte within the at least one discrete given area of said surface is accordingly less than about 20%.

Also in this second aspect, the present invention is directed to a method of manufacturing a microarray comprising the steps of: (i) providing a surface to which are bound a plurality of reactive groups, wherein only approximately 20% or less of said reactive groups are able to react at any one time; (ii) optionally contacting said reactive groups with a linking moiety under conditions whereby said linking moiety reacts with one or more reactive groups which is able to react, resulting in the binding of the linking moiety to said surface; and (iii) depositing a sample of an analyte on the surface within at least one discrete given area of the surface such that the analyte is immobilised on said surface via direct binding to one or more reactive groups which is able to react, or via indirect binding via said linking moiety to a reactive group; wherein the surface coverage of the bound analyte within the at least one given area of the surface is accordingly less than about 20%.

In an embodiment of this second aspect of the invention, the microarray is a low density microarray.

In the context of all the embodiments of this invention, e.g. this second aspect of the invention, the conditions which impact the ability of the linking moiety to react with one or more reactive groups, or which impact the ability of the analyte to react directly with one or more reactive groups, can be varied in order to vary the surface density/coverage of linking moiety and/or analytes on the surface of the microarray and thus achieve the low surface density/coverage of the present invention. Such conditions include: i) concentration of the solution of linking moiety (e.g. streptavidin) or the concentration of analyte which is applied to the reactive groups; ii) the length of time of the incubation of the reactive groups with the analyte or with the solution of linking moiety (e.g. streptavidin); iii) the temperature of incubation of the reactive groups with the analyte or with the solution of linking moiety (e.g. streptavidin); iv) the pH of incubation of the reactive groups with the analyte or with the solution of linking moiety (e.g. streptavidin); v) the conditions under which the microarray slide has been stored/equilibrated before immobilisation of analyte or exposure to the linking moiety (e.g. streptavidin), which can result in the passive hydrolysis of some of the reactive groups (e.g. NHS esters) with time and vi) co-incubation of linking moiety (e.g. streptavidin) or analyte with defined molar ratios of competing molecules that can compete for reaction with the reactive groups (but which are themselves not linking moieties), for example, co-incubation with bovine serum albumin, milk powder, casein hydrolysate, ethanolamine, glycine, a chemically-reactive nucleic acid polymer or nucleic acid sugar. Appropriate combinations of conditions can be selected in order to bind particular analytes or linking moieties (e.g. streptavidin) to the reactive groups at an appropriate surface density/coverage. Typical conditions may include: i) a concentration of the linking moiety (e.g. streptavidin) of 0.01 to 0.9 mg/ml or 0.1 to 10 mg/ml, for example 0.05 to 0.5 mg/ml, 0.05 to 0.3 mg/ml, 0.05 to 0.2 mg/ml, 0.01 to 0.2 mg/ml, about 0.08 mg/ml, about 0.1 mg/ml or about 0.25 mg/ml, 0.2 to 5 mg/ml, 0.5 to 2 mg/ml, 0.8 to 1.2 mg/ml or about 1 mg/ml; ii) an incubation time of 1 min to 24 hours, for example 10 min to 12 hours, 15 min to 5 hours, 20 min to 2 hours, 30 min to 1.5 hours or about 10 minutes, 20 minutes, 30 minutes or 1 hour; iii) an incubation temperature of 4 °C to 50 °C, for example 4 °C to 15 °C, 10 °C to 40 °C, 15 °C to 30 °C or about 4 °C or about 20 °C. (room temperature) iv) an incubation pH of 6 to 10, for example 6.5 to 9.5, 7 to 9, 8 to 9 or about 8.5; and iv) storage/equilibration of the microarray slide according to manufacturers' instructions and observing the recommended use by date, or using a microarray slide that is beyond its recommended use by date, or which has been stored, e.g. at −20 °C, for at least 3 months, at least 6 months, at least 1 year, at least 18 months or at least 2 years before use. Furthermore, prior to binding of analyte, active chemical steps may also be taken to hydrolyse or preserve reactive groups (e.g. NHS esters). Such active chemical steps are well-known to a person of skill in the art.

In an embodiment of this second aspect of the invention, step (ii) is not optional. In an embodiment of this second aspect of the invention, step (ii) is not optional, said linking moiety is a biotin-binding protein as described elsewhere herein, and said analyte is biotinylated. In an embodiment of this second aspect of the invention, step (ii) is not optional, said linking moiety is a biotin-binding protein as described elsewhere herein, said analyte is biotinylated, and said method comprises a further step (iv) following step (iii), in which a solution of biotin (i.e. free biotin which is not bound to analyte) is applied to the surface of the microarray, such that biotin binding sites on said linking moiety which are not bound to biotinylated analyte are occupied by biotin.

Accordingly, in an embodiment of this second aspect of the invention, there is provided a method of manufacturing a microarray comprising the steps of: (i) providing a surface to which are bound a plurality of reactive groups, wherein only approximately 20% or less of said reactive groups are able to react at any one time; (ii) contacting said reactive groups with a linking moiety under conditions whereby said linking moiety reacts with one or more reactive groups which is able to react, resulting in the binding of the linking moiety to said surface; (iii) depositing a sample of a biotinylated analyte on the surface within at least one discrete given area of the surface such that the analyte is immobilised on said surface via indirect binding via said linking moiety to a reactive group; wherein the surface density of the bound analyte within the at least one given area of said surface is accordingly less than about 20% and thereafter (iv) applying a solution of biotin to the surface of the microarray.

In a further embodiment of this second aspect of the invention, there is provided a method of manufacturing a microarray comprising the steps of:
(i) providing a surface to which are bound a plurality of reactive groups, wherein only approximately 20% or less of said reactive groups are able to react at any one time;
(ii) contacting said reactive groups with a linking moiety which is a biotin-binding molecule which is selected from the group consisting of: (a) an anti-biotin antibody; (b) avidin; (c) neutravidin; (d) streptavidin, and (e) a fragment, variant or analogue of streptavidin, avidin, neutravidin or said anti-biotin antibody which retains biotin-binding capability, wherein said contact is made under conditions whereby said linking moiety reacts with one or more reactive groups which is able to react, resulting in the binding of the linking moiety to said surface;
(iii) contacting the microarray with a sample of a biotinylated polypeptide, wherein biotin is bound to said polypeptide via a biotin carboxyl carrier protein (BCCP) motif or an Avi-tag (SEQ ID NO: 1), such that the polypeptide is immobilised on said surface via indirect binding via said linking moiety to a reactive group; wherein the surface density of the bound polypeptide within a given area of said surface is accordingly less than 20%; and thereafter

(iv) applying a solution of biotin to the surface of the microarray.

In an embodiment of this second aspect of the invention, there is provided a method of manufacturing a microarray comprising the steps of: (i) providing a surface to which are bound a plurality of reactive groups, wherein only approximately 20% or less of said reactive groups are able to react at any one time; (ii) contacting said reactive groups with a linking moiety under conditions whereby said linking moiety reacts with one or more reactive groups which is able to react, resulting in the binding of the linking moiety to said surface; (iii) depositing a sample of a biotinylated analyte on the surface within at least one discrete given area of the surface such that the analyte is immobilised on said surface via indirect binding via said linking moiety to a reactive group; wherein the surface coverage of the bound analyte within the at least one discrete given area of said surface is accordingly less than 20% and thereafter (iv) applying a solution of biotin to the surface of the microarray.

In a further embodiment of this second aspect of the invention, there is provided a method of manufacturing a microarray comprising the steps of:
(i) providing a surface to which are bound a plurality of reactive groups, wherein only approximately 20% or less of said reactive groups are able to react at any one time;
(ii) contacting said reactive groups with a linking moiety which is a biotin-binding molecule which is selected from the group consisting of: (a) an anti-biotin antibody; (b) avidin; (c) neutravidin; (d) streptavidin, and (e) a fragment, variant or analogue of streptavidin, avidin, neutravidin or said anti-biotin antibody which retains biotin-binding capability, wherein said contact is made under conditions whereby said linking moiety reacts with one or more reactive groups which is able to react, resulting in the binding of the linking moiety to said surface;
(iii) depositing a sample of a biotinylated polypeptide on the surface within at least one discrete given area of the surface, wherein biotin is bound to said polypeptide via a biotin carboxyl carrier protein (BCCP) motif or an Avi-tag (SEQ ID NO: 1), such that the polypeptide is immobilised on said surface via indirect binding via said linking moiety to a reactive group; wherein the surface coverage of the bound polypeptide within the at least one given area of said surface is accordingly less than about 20%; and thereafter
(iv) applying a solution of biotin to the surface of the microarray.

In an additional embodiment of this second aspect of the invention, said reactive groups are bound to said surface via a hydrophilic organic polymer, which may be selected from the group consisting of polyacrylamide, polyurethane, polyethyleneimine and polyethylene glycol, for example polyethylene glycol.

In the preceding embodiments, where reference is made to the application of a solution of biotin to the surface of the microarray, the solution of biotin is applied to the surface at a concentration of between 10 and 100 μM, for example between 25 and 75 μM, for example between 25 and 75 μM, for example about 50 μM.

In a third aspect, the present invention is directed to a method of reducing the density of analyte bound to the surface of a microarray to form a low density (or low surface coverage) microarray comprising the steps of (i) providing a surface to which are bound a plurality of reactive groups; (ii) causing or allowing a portion of said reactive groups to be deactivated or inaccessible such that approximately 20% or less of said reactive groups are able to react at any one time; (iii) optionally contacting said reactive groups with a linking moiety under conditions whereby one or more reactive groups retaining reactivity are able to react with the linking moiety resulting in the binding of the linking moiety to said surface; and (iv) contacting the microarray with a sample of an analyte such that the analyte is immobilised on said surface via direct binding to one or more reactive groups, or via indirect binding via said linking moiety to one or more reactive groups; wherein the surface density (or surface coverage) of the bound analyte within a given area of said surface is accordingly less than 20%.

In a fourth aspect, the present invention is directed to a method of:
i) increasing the analyte signal to background noise ratio of a microarray;
ii) increasing the rotational and conformational freedom of an analyte immobilised on a microarray; and/or
iii) increasing the proportion of physiologically-relevant interactions between analytes immobilised on a microarray and test molecules applied to said microarray;
wherein said method comprises the step of providing a microarray comprising a surface to which are bound a plurality of reactive groups, wherein the analyte is immobilised on said surface via direct binding to one or more of the reactive groups or via indirect binding via a linking moiety to one or more of the reactive groups, and wherein the surface density (or surface coverage) of the analyte immobilised within a given area of said surface is less than 20%.

In an embodiment of this fourth aspect of the invention, the microarray is a low density microarray. In an embodiment of this fourth aspect of the invention, the microarray is a low coverage microarray.

In an embodiment of this fourth aspect of the invention, the increase in signal to noise ratio achieved is a function of (i) increased physiologically-relevant signal and (ii) decreased non-specific (i.e. non-physiologically-relevant) background. Without being bound by theory, it is thought that non-specific background binding is reduced because the low surface density (or low surface coverage) of analytes minimises non-specific aggregation of the immobilised analytes. Any such aggregation would promote non-specific binding of test molecules (which are applied to the microarray to determine whether they specifically bind to the immobilised analytes) to those aggregates. Non-specific background binding is also reduced to non-aggregated analytes by reducing molecular crowding effects (which promote non-specific interactions). Furthermore, it is thought that specific binding is increased because the increased spacing between immobilised antigen molecules, combined with increased rotational and conformational freedom of the immobilised analyte molecules, enables a higher proportion of physiologically-relevant integrations with test molecules (which are applied to the microarray to determine whether they specifically bind to the immobilised analytes).

The features of the embodiments of the first aspect of the invention (and, where appropriate, the definitions of features discussed in the context of the first aspect of the invention), may be combined with or applied to the features of the embodiments of the second, third or fourth aspects of the invention. Thus, for example, the features of the microarray described in the context of the first aspect of the invention may equally be applied to the embodiments of the methods of the second, third or fourth aspects of the invention.

The invention further provides a microarray preparable by the embodiments of the methods of the second, third or fourth aspects of the invention.

In a fifth aspect, the present invention is directed to the use of a surface of a microarray as a low density (or low coverage) surface on which the surface density (or surface coverage) of analyte immobilised within a given area of said surface is less than 20% and wherein the analyte is optionally immobilised on said microarray via a linking moiety.

In a sixth aspect, the present invention is directed to the use of a surface for the manufacture of a low density (or low coverage) microarray comprising immobilised analyte, wherein the surface density (or surface coverage) of analyte immobilised within a given area of said surface is less than 20% and wherein the analyte is optionally immobilised on said microarray via a linking moiety.

In a seventh aspect, the present invention is directed to the use of reactive groups of a surface suitable for forming a low density (or low coverage) microarray for reducing the density of analyte immobilised on said surface, wherein a proportion of said reactive groups are unable to react with said analyte such that the surface density (or surface coverage) of analyte immobilised on a given area of said surface is less than 20%, and wherein said analyte is optionally immobilised on said surface via linking moiety.

In an eighth aspect, the present invention is directed to the use of reactive groups of a surface suitable for forming a low density (or low coverage) microarray for the manufacture of a low density (or low coverage) protein microarray, wherein a proportion of said reactive groups on said surface are unable to react with an analyte such that the surface density (or surface coverage) of analyte immobilised on a given area of said surface is less than 20%, and wherein said analyte is optionally immobilised on said surface via a linking moiety.

The features of the embodiments of the first aspect of the invention (and, where appropriate, the definitions of features discussed in the context of the first aspect of the invention), may be combined with or applied to the features of the embodiments of the fifth, sixth, seventh or eighth aspects of the invention. Thus, for example, the features of the microarray described in the context of the first aspect of the invention may equally be applied to the embodiments of the uses of the fifth, sixth, seventh or eighth aspects of the invention.

In a ninth aspect, the present invention is directed to the use of a microarray as defined in the first aspect for:
the identification of interactions between said analyte and test molecules applied to said analyte;
the determination of an antibody profile of a subject;
identifying a biomolecule which specifically binds to said immobilised analyte;
the identification of an antibody which specifically binds said immobilised analyte and which is suitable for the diagnosis or treatment of a disease; or
identifying a biomolecule which specifically binds said immobilised analyte and which is capable of treating a disease mediated by said immobilised analyte.

In a tenth aspect of the invention, there is provided a method of manufacturing a microarray comprising the steps of: (i) providing a surface to which are bound a plurality of reactive groups; (ii) contacting said reactive groups with a linking moiety comprising a biotin-binding molecule under conditions whereby said linking moiety reacts with one or more reactive groups which is able to react, resulting in the binding of the linking moiety to said surface; (iii) contacting the microarray with a sample of a biotinylated analyte such that the analyte is immobilised on said surface via indirect binding via said linking moiety to a reactive group; and thereafter (iv) applying a solution of biotin to the surface of the microarray.

In an eleventh aspect of the invention, there is provided a method of increasing the analyte signal to background noise ratio of a microarray comprising the steps of: (i) providing a surface to which are bound a plurality of reactive groups, wherein only approximately 20% or less of said reactive groups are able to react at any one time; (ii) contacting said reactive groups with a linking moiety, which is a biotin-binding moiety, under conditions whereby said linking moiety reacts with one or more reactive groups which is able to react, resulting in the binding of the linking moiety to said surface; (iii) depositing a sample of a biotinylated analyte on the surface within at least one discrete given area of the surface such that the analyte is immobilised on said surface via indirect binding via said linking moiety to a reactive group; wherein the surface coverage (or surface density) of the bound analyte within the at least one discrete given area of said surface is accordingly less than about 20% and thereafter (iv) applying a solution of biotin to the surface of the microarray.

In a further embodiment of this eleventh aspect of the invention, there is provided a method of increasing the analyte signal to background noise ratio of a microarray comprising the steps of:
(i) providing a surface to which are bound a plurality of reactive groups, wherein only approximately 20% or less of said reactive groups are able to react at any one time;
(ii) contacting said reactive groups with a linking moiety which is a biotin-binding molecule which is selected from the group consisting of: (a) an anti-biotin antibody; (b) avidin; (c) neutravidin; (d) streptavidin, and (e) a fragment, variant or analogue of streptavidin, avidin, neutravidin or said anti-biotin antibody which retains biotin-binding capability, wherein said contact is made under conditions whereby said linking moiety reacts with one or more reactive groups which is able to react, resulting in the binding of the linking moiety to said surface;
(iii) depositing a sample of a biotinylated polypeptide on the surface within at least one given area of the surface, wherein biotin is bound to said polypeptide via a biotin carboxyl carrier protein (BCCP) motif or an Avi-tag (SEQ ID NO: 1), such that the polypeptide is immobilised on said surface via indirect binding via said linking moiety to a reactive group; wherein the surface coverage (or surface density) of the immobilised polypeptide within the at least one discrete given area of said surface is accordingly less than about 20%; and thereafter
(iv) applying a solution of biotin to the surface of the microarray.

In an additional embodiment of this eleventh aspect of the invention, said reactive groups are bound to said surface via a hydrophilic organic polymer, which may be selected from the group consisting of polyacrylamide, polyurethane, polyethyleneimine and polyethylene glycol, for example polyethylene glycol.

In the embodiments of this invention, where reference is made to the application of a solution of biotin to the surface of the microarray, the solution of biotin is applied to the surface at a concentration of between 10 and 100 μM, for example between 25 and 75 μM, for example between 25 and 75 μM, for example about 50 μM.

The features of the embodiments of the first aspect of the invention (and, where appropriate, the definitions of features discussed in the context of the first aspect of the invention), may be combined with or applied to the features of the embodiments of the tenth, eleventh, twelfth or thirteenth aspects of the invention. Thus, for example, the features of the microarray described in the context of the first aspect of the invention may equally be applied to the embodiments of the methods of the tenth, eleventh or thirteenth aspects of the invention, or the embodiments of the microarrays of the twelfth aspect of the invention.

In a twelfth aspect of the invention, there is provided a microarray comprising a surface to which are bound a plurality of reactive groups, wherein an analyte is immobilised on said surface via direct binding to one or more of the reactive groups or via indirect binding via a linking moiety to one or more of the reactive groups, and wherein the number density of said analyte within at least one discrete given area of said surface is less than about 300 per micrometre squared.

In an embodiment of this twelfth aspect, the number density of immobilised analyte molecules is fewer than about 275 per micrometre squared, e.g. fewer than about 250 per micrometre squared, fewer than about 225 per micrometre squared, fewer than about 200 per micrometre squared, fewer than about 175 per micrometre squared, fewer than about 150 per micrometre squared, fewer than about 125 per micrometre squared, fewer than about 100 per micrometre squared, fewer than about 75 per micrometre squared, fewer than about 50 per micrometre squared, fewer than about 25 per micrometre squared, fewer than about 20 per micrometre squared, fewer than about 15 per micrometre squared, fewer than about 10 per micrometre squared or fewer than about 5 per micrometre squared, optionally when analysed by AFM or optionally when analysed SEM. By number density is meant the number of analyte molecules immobilised on said surface per unit area.

In a thirteenth aspect of the invention, there is provided a method of manufacturing a microarray comprising the steps of: (i) providing a surface to which are bound a plurality of reactive groups; (ii) contacting said reactive groups with a solution of a linking moiety wherein the concentration of the linking moiety in said solution is less than 1 mg/ml and whereby said linking moiety reacts with one or more reactive groups which is able to react, resulting in the binding of the linking moiety to said surface. In this context, binding may mean a covalent linkage. Alternatively, binding may mean a non-covalent linkage, e.g. the interaction between an antibody and an antigen, or between a ligand and a receptor.

In an embodiment of this thirteenth aspect of the invention, the method comprises a further step (iii) of depositing a sample of an analyte on the surface such that the analyte is immobilised on said surface via indirect binding via said linking moiety to the one or more reactive groups. In an embodiment of this thirteenth aspect of the invention, the concentration of said linking moiety, for example streptavidin, in said solution is 0.8 mg/ml or less, 0.6 mg/ml or less, 0.5 mg/ml or less, 0.4 mg/ml or less, 0.3 mg/ml or less, 0.25 mg/ml or less, 0.24 mg/ml or less, 0.2 mg/ml or less, 0.15 mg/ml or less, 0.1 mg/ml or less, 0.05 mg/ml or less or 0.01 mg/ml or less. In an embodiment of this thirteenth aspect of the invention, the concentration of said linking moiety, for example streptavidin, in said solution is about 0.5 mg/ml, about 0.25 mg/ml, about 0.125 mg/ml, about 0.1 mg/ml, about 0.08 mg/ml, about 0.06 mg/ml, about 0.03 mg/ml or about 0.01 mg/ml. In an embodiment of this thirteenth aspect of the invention, the linking moiety is a biotin-binding molecule. In an embodiment of this thirteenth aspect of the invention, said linking moiety is a biotin-binding molecule and said analyte is biotinylated. In an embodiment of this thirteenth aspect of the invention, said biotin-binding molecule is a protein. In an embodiment of this thirteenth aspect of the invention, said biotin-binding molecule is selected from the group consisting of (i) an anti-biotin antibody; (ii) avidin; (iii) neutravidin; (iv) streptavidin, or (iii) a fragment, variant or analogue of streptavidin, avidin, neutravidin or said anti-biotin antibody which retains biotin-binding capability. In an embodiment of this thirteenth aspect of the invention, said biotin-binding molecule comprises a sequence having at least 80% sequence identity, preferably at least 90% sequence identity, more preferably 100% sequence identity to SEQ ID NO. 3. In an embodiment of this thirteenth aspect of the invention, said biotin-binding molecule, e.g. streptavidin, is a tetramer (i.e. is in tetrameric form) and is not aggregated.

In an embodiment of this thirteenth aspect of the invention, said analyte comprises a tag which permits immobilisation of said analyte on said surface. In an embodiment of this thirteenth aspect of the invention, said analyte comprises one or more of a polypeptide, a nucleic acid, a lipid and a carbohydrate. In an embodiment of this thirteenth aspect of the invention, said analyte comprises one or more of a polypeptide and a nucleic acid. In an embodiment of this thirteenth aspect of the invention, said analyte is a polypeptide, for example a glycoprotein. In an embodiment of this thirteenth aspect of the invention, said analyte is a polypeptide. In an embodiment of this thirteenth aspect of the invention, said analyte is a polypeptide, and said tag which permits immobilisation of said polypeptide on said surface is fused to the N- or the C-terminus of the polypeptide. In an embodiment of this thirteenth aspect of the invention, said polypeptide is correctly folded. In an embodiment of this thirteenth aspect of the invention, said analyte is biotinylated. In an embodiment of this thirteenth aspect of the invention, said biotinylated analyte is chemically biotinylated or enzymatically biotinylated. In an embodiment of this thirteenth aspect of the invention, said tag which permits immobilisation of said analyte on said surface comprises a biotin carboxyl carrier protein (BCCP) motif or an Avi-tag (SEQ ID NO: 1). In an embodiment of this thirteenth aspect of the invention, said BCCP motif has at least 80% sequence identity, preferably at least 90% sequence identity, more preferably 100% sequence identity to SEQ ID NO. 2. In an embodiment of this thirteenth aspect of the invention, said BCCP motif is correctly folded. In an embodiment of this thirteenth aspect of the invention, the biotin is bound to: (i) a biotin attachment domain within said BCCP motif or (ii) said Avi-tag. In an embodiment of this thirteenth aspect of the invention, biotin is attached to said biotin attachment domain or said Avi-tag via enzymatic biotinylation, preferably via biotin ligase.

In an embodiment of this thirteenth aspect of the invention, the analyte is capable of binding to the linking moiety with a K_{D} of less than about 1×10⁻¹² mol/L, for example less than about 1×10⁻¹³ mol/L, for example about 1×10⁻¹⁴ mol/L. In an embodiment of this thirteenth aspect of the invention, said analyte comprises a biotin carboxyl carrier protein (BCCP) motif or an Avi-tag (SEQ ID NO: 1).

In an embodiment of this thirteenth aspect of the invention, the method comprises a further step (iv) of applying a solution of a blocking agent to the surface of the microarray. In an embodiment of this thirteenth aspect of the invention, said blocking agent is selected from the group consisting of skimmed milk powder, bovine serum albumin (BSA), casein, polyethylene glycol (PEG), polyvinylpyrrolidone (PVP), gelatin, serum (e.g. fetal calf serum), and biotin. In an embodiment of this thirteenth aspect of the invention, said blocking agent is able to bind to the linking moiety with a K_{D} of less than about 1×10⁻¹² mol/L, for example less than about 1×10⁻¹³ mol/L, for example about 1×10⁻¹⁴ mol/L. In an embodiment of this thirteenth aspect of the invention, said blocking agent is biotin. In an embodiment of this thirteenth aspect of the invention, the concentration of the blocking agent, e.g. biotin, in the applied solution is between 1 mM and 100 mM, e.g. between 10 mM and 80 mM, e.g. between 20 mM and 70 mM, e.g. between 40 mM and 60 mM, e.g. about 50 mM.

In an embodiment of this thirteenth aspect of the invention, said biotin-binding molecule is selected from the group consisting of (i) an anti-biotin antibody; (ii) avidin; (iii) neutravidin; (iv) streptavidin, or (iii) a fragment, variant or analogue of streptavidin, avidin, neutravidin or said anti-biotin antibody which retains biotin-binding capability. In an embodiment of this thirteenth aspect of the invention, said biotin-binding molecule comprises a sequence having at least 80% sequence identity, preferably at least 90% sequence identity, more preferably 100% sequence identity to SEQ ID NO. 3.

In an embodiment of this thirteenth aspect of the invention, said reactive groups on said surface are selected from carboxylic acid groups, activated carboxylic acid groups, amine groups, imidoester groups, maleimide groups, haloacetyl groups, pyridyl dithiol groups, azide groups, hydrazide groups, alkoxyamine groups, thiol groups, aryl azide groups and diazirine groups. In an embodiment of this thirteenth aspect of the invention, said reactive groups on said surface are selected from carboxylic acid groups, activated carboxylic acid groups, amine groups and maleimide groups. In an embodiment of this thirteenth aspect of the invention, said reactive groups on said surface are activated carboxylic acid groups, for example 1-ethyl-3-(3-dimethylaminopropyl) carbodiimide (EDC) activated or N-hydroxysuccinimide (NHS) activated carboxylic acid groups. In an embodiment of this thirteenth aspect of the invention, said reactive groups are bound to said surface via a hydrophilic organic polymer. In an embodiment of this thirteenth aspect of the invention, said hydrophilic organic polymer is selected from the group consisting of polyacrylamide, polyurethane, polyethyleneimine and polyethylene glycol, preferably polyethylene glycol. In an embodiment of this thirteenth aspect of the invention, said polyethylene glycol has an average molecular weight in the range 500 to 20000.

In an embodiment of this thirteenth aspect of the invention, said solution of a linking moiety, e.g. streptavidin, further comprises a competitor molecule which is able to compete with the linking moiety for reaction with the reactive groups. In an embodiment of this thirteenth aspect of the invention, said competitor molecule comprises a free amine group. In an embodiment of this thirteenth aspect of the invention, said competitor molecule is selected from the group consisting of an amino acid and an alkanolamine. In an embodiment of this thirteenth aspect of the invention, said competitor molecule is selected from the group consisting of glycine, alanine, serine, lysine, arginine, histidine, valine, isoleucine, leucine, phenylalanine, tyrosine, tryptophan, methanolamine and ethanolamine. In an embodiment of this thirteenth aspect of the invention, said competitor molecule is selected from the group consisting of glycine and ethanolamine. In an embodiment of this thirteenth aspect of the invention, said competitor molecule is glycine. In an embodiment of this thirteenth aspect of the invention, the competitor molecule is present in said solution of a linking moiety, e.g. streptavidin, at a concentration of between 1 mM and 100 mM, e.g. between 10 mM and 80 mM, e.g. between 20 mM and 70 mM, e.g. between 40 mM and 60 mM, e.g. about 50 mM.

In an embodiment of this thirteenth aspect of the invention, the pH of the solution of the linking moiety, e.g. streptavidin, is about 7.5 to about 11, for example about 8 to about 9.5 or about 8.5 to about 9, for example about 8.5. In an embodiment of this thirteenth aspect of the invention, the pH of the solution of the linking moiety, e.g. streptavidin, is about 4 to about 7.5, for example about 4.5 to 6, for example about 4.5.

In an embodiment of this thirteenth aspect of the invention, said reactive groups are contacted with the solution of the linking moiety (e.g. streptavidin) for a limited time period (AKA the “incubation time”). In an embodiment of this thirteenth aspect of the invention, the incubation time is from 1 min to 24 hours, for example 10 min to 12 hours, 15 min to 5 hours, 20 min to 2 hours, 30 min to 1.5 hours or about 10 minutes, 20 minutes, 30 minutes or 1 hour. In an embodiment of this thirteenth aspect of the invention, the incubation time is less than 1 hour, e.g. about 45 minutes.

In an embodiment of this thirteenth aspect of the invention, said reactive groups are contacted with the solution of the linking moiety (e.g. streptavidin) at a certain temperature (AKA the “incubation temperature”). In an embodiment of this thirteenth aspect of the invention, the incubation temperature is from 4 °C to 50 °C, for example 4 °C to 15 °C, 10 °C to 40 °C, 15 °C to 30 °C or about 4 °C or about 20 °C (room temperature).

In an embodiment of this thirteenth aspect of the invention, the sample of the analyte is deposited on the surface in the form of a solution, e.g. an aqueous solution. In an embodiment of this thirteenth aspect of the invention, the solution of analyte is diluted, e.g. in a suitable aqueous buffer before deposition on the surface, for example at least a 1 in 2 dilution, at least a 1 in 3 dilution, at least a 1 in 4 dilution, at least a 1 in 5 dilution, at least a 1 in 10 dilution, at least a 1 in 20 dilution, at least a 1 in 25 dilution, at least a 1 in 50 dilution or at least a 1 in 100 dilution.

In an embodiment of this thirteenth aspect of the invention, the method results in a density of said linking moiety within at least one discrete given area of said surface of fewer than about 300 linking moieties per micrometre squared, for example fewer than about 275 per micrometre squared, fewer than about 250 per micrometre squared, fewer than about 225 per micrometre squared, fewer than about 200 per micrometre squared, fewer than about 175 per micrometre squared, fewer than about 150 per micrometre squared, fewer than about 125 per micrometre squared, fewer than about 100 per micrometre squared, fewer than about 75 per micrometre squared, fewer than about 50 per micrometre squared fewer than about 25 per micrometre squared, fewer than about 20 per micrometre squared, fewer than about 15 per micrometre squared, fewer than about 10 per micrometre squared or fewer than about 5 linking moieties per micrometre squared.

In an embodiment of this thirteenth aspect of the invention, the method results in a density of said analyte within at least one discrete given area of said surface of fewer than about 300 analyte molecules per micrometre squared, for example fewer than about 275 per micrometre squared, fewer than about 250 per micrometre squared, fewer than about 225 per micrometre squared, fewer than about 200 per micrometre squared, fewer than about 175 per micrometre squared, fewer than about 150 per micrometre squared, fewer than about 125 per micrometre squared, fewer than about 100 per micrometre squared, fewer than about 75 per micrometre squared, fewer than about 50 per micrometre squared fewer than about 25 per micrometre squared, fewer than about 20 per micrometre squared, fewer than about 15 per micrometre squared, fewer than about 10 per micrometre squared or fewer than about 5 analyte molecules per micrometre squared.

In a fourteenth aspect of the invention, the invention provides for a microarray obtainable by any of the methods of the tenth or thirteenth aspects of the invention.

Whilst the present invention has been described and illustrated with reference to particular embodiments, it will be appreciated by those of ordinary skill in the art that the invention lends itself to many different variations not specifically illustrated herein.

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

### EXAMPLE SECTION

### Example 1: Manufacture of a streptavidin-coated microarray slide

Microarray fabrication is carried out using streptavidin-coated, long-chain polyethylene gycol (PEG) based glass slides (Nexterion H slides (Schott, Germany)). The PEG hydrogel coating acts as a long, flexible spacer to allow for a significant level of rotational and conformational freedom of immobilised analytes such as proteins, thus enabling quantitative measurement of biomolecular interactions with the immobilized analytes. In addition, PEG is considerably superior to proteinaceous blocking agents such as bovine serum albumin or milk powder in reducing the non-specific background in surface-based assays. The polymeric structure of PEG further creates an aqueous-like environment resembling unstructured water, thus helping to retain water and keep immobilised proteins folded and functional.

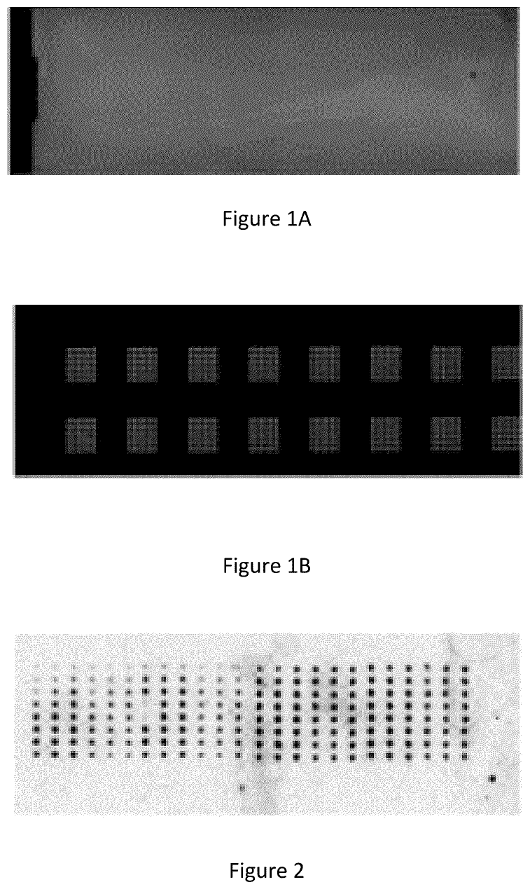

The initial step of preparing the microarray surfaces involves the coating of hydrogel PEG slides with a uniform, low density (<10% surface coverage) layer of streptavidin. Multiple hydrogel slides per batch can be coated with streptavidin in parallel to reduce coating variability (typically less than 15%). Quality control testing of the coating variability amongst slides within the batch is done by incubating a coated slide with biotinylated bovine serum albumin (BSA) conjugated with a fluorescent dye (Cy5), and coefficient of variations are then measured from data extrapolated from fluorescent intensities across the entire surface (see FIG. 1A).

For homogenous slide coating, the Nexterion H slides were equilibrated at room temperature before opening the packaging. The batch processed procedure started with the incubation of the N-hydroxysuccinimide (NHS)-activated Nexterion H slides with 1 mg/ml streptavidin (PROSPEC, pro-283) solution in HEPES buffer (200 mM KCL, 0.02% Triton X-100, 50 mM HEPES, pH 8.5) for 1 hr at room temperature, followed by a blocking step in ethanolamine (50 mM ethanolamine in 50 mM potassium phosphate buffer pH 8.0) for an additional hour. The slides were then washed 3 times in 100 mM potassium phosphate buffer (100 mM K₂PO₄ (94% K₂HPO₄, 6% KH₂PO₄, pH8.0)) and once in dH2O followed by drying by centrifugation at 2000 g for 5 mins. Coated slides were then stored dry at −20 °C in a desiccated environment until use for microarray fabrication.

### Example 2: Expression of CYP450 Protein with BCCP Tag and Subsequent Biotinylation

Recombinant proteins used for array fabrication are expressed as a fusion to a compact folded ~80-residue domain derived from E. coli called biotin carboxyl carrier protein (BCCP) (Athappilly, F. K. & Hendrickson, W. A., Structure 3, 1407-19 (1995) & Chapman-Smith, A. & Cronan, J. E., Trends Biochem. Sci. 24, 359-63 (1999)). The BCCP domain consists of amino acids 74-156 of the E. coli AccB protein, and is disclosed herein as SEQ ID NO: 2. The BCCP domain is fused at the C-terminus of the CYP450 protein. A His tag is fused at the C-terminus of the BCCP domain.

### Expression in Insect Cells

Synthesized genes of target proteins were cloned into a proprietary E. coli/Spodoptera frugiperda transfer vector, pPRO8 or pRO30, such that the construct encoded the full-length target protein as an in-frame fusion to three consecutive tags as follows: a Biotin Carboxyl Carrier Protein (BCCP); a c-Myc tag; and a hexahistidine tag. pPRO8 is used for generating fusion proteins where the BCCP-cmyc is fused at the C-terminal of the antigen. For pRO30 the tag is fused at the N-terminal domain of the antigen, where the hexahistidine tag is followed by c myc and BCCP. If required, a signal peptide for guiding proteins to the secretory pathway was included between the hexahistidine and c-Myc tag in pR030 vectors.

pPRO8 and pRO30 consist of the viral polyhedrin promoter and cloning sites. Flanking this polh-BCCP expression cassette are the baculoviral 603 gene and the 1629 genes to enable subsequent homologous recombination of the construct into a replication-deficient baculoviral genome (Blackburn, Shoko, & Beeton-Kempen, 2012).

Following co-transfection of S. frugiperda Sf9 cells with a relevant transfer vector plus a linearized, replication deficient bacmid vector (Autographa californica baculovirus vector pBAC10: KO1629), baculovirus was amplified and recombinant proteins were expressed in S. frugiperda superSf9-3 strain (Oxford Expression Technologies, Oxford, UK).

3 mL of Spodoptera frugiperda Sf9 cells were cultured in Sf-900 III SFM medium (Gibco) expressing individual C-terminally BCCP-tagged recombinant human CYP450 enzymes (CYP3A4, CYP2C9, CYP2D6). The cells were cultured at 27 °C for 3 days. The BCCP tag is cross-recognized by insect cell biotin ligases enabling it to be biotinylated efficiently without the need to co-express the E. coli biotin ligase. Clarified cell lysates were prepared in insect lysis buffer (25 mM Hepes, 50 mM KCL, 20% glycerol, 0.1% Triton X100, 1×Halt™ Protease Inhibitor Cocktail, EDTA-free (Thermo Scientific, Waltham, MA, USA), 0.25% sodium deoxycholate acid, Pierce Universal nuclease (Thermo Scientific), pH 8.5). Expression yields and in vivo biotinylation of each antigen were assessed by Western blot using a streptavidin-HRP conjugate probe (GE Healthcare, Chicago, IL, USA). Crude lysates were diluted 1:1 with 40% (w/v) sucrose in PBS and were stored at −80 °C before microarray printing.

### Expression in Bacterial Cells

For the recombinant expression of target proteins in E. coli, synthesized genes were cloned into either pMDOO4 or pAN101. pMDOO4 is for the expression of N-terminal hexahistidine BCCP fusion proteins, whereas pAN101 is for the C-terminal BCCP-hexahistidine fusion protein. Expression is carried out via the T5 promoter/lac operator element using IPTG induction or lactose/glucose autoinductive conditions. E. coli cells are lysed using bacterial lysis buffer (Bugbuster) and in vivo biotinylation is assessed as described earlier. Clarified lysates are stored in 20% glycerol at −80 °C before microarray printing.

### Example 3: Biotinylation of Peptides, Nucleic Acids, Carbohydrates or Lipids

### Peptide Biotinylation

Biotinylated peptides are commercially available. During Fmoc solid-phase synthesis, peptides are synthesized with a biotin moiety linked to their N-terminus. Simultaneously, a C6 spacer is incorporated between the biotin moiety and the peptide to avoid downstream steric interference with the biotin-streptavidin interaction. Alternatively, the spacer can consist of a hydrocarbon chain (e.g. via aminohexanoic acid) or polyethylene oxide. The latter has the added advantage of improving solubility.

### Nucleic Acid Biotinylation

Biotinylated DNA is commercially available. Single stranded (ss) DNA is synthesized with a biotin moiety at either the 5′ or 3′ end. A C6 spacer is inserted between the biotin moiety and the DNA sequence to avoid downstream steric interference with the biotin-streptavidin interaction. Alternatively, a triethyleneglycol (TEG) spacer increases the oligoDNA-biotin distance to 15 atoms. In the case of double strand DNA, the complementary strand is annealed to an already-biotinylated ssDNA molecule.

### Carbohydrate Biotinylation

(a) Commercial kits are available to biotinylate carbohydrate molecules. Carbohydrate chain oxidization to aldehydes facilitates their biotinylation. For example, sodium meta-periodate (NaIO₄) oxidizes sialic acid—commonly found at glycan chain termini—to form an aldehyde susceptible to alkoxyamines linked to biotin and a spacer (e.g. commercially-available alkoxyamine-PEG spacer-biotin).
(b) Reactive biotin-LC-hydrazine can readily be coupled to the reducing end of any carbohydrate (e.g. oligosaccharide) chain, without disturbing glycan ability to cognitively bind lectin partners (Grün et al., 2006, Analytical Biochemistry, 354 (1), 54-63, https://doi.org/10.1016/j.ab.2006.03.055).
(c) Poly(2-methylacrylic acid) (pMAA)—which possesses multiple carboxyl groups for multivalent carbohydrate-binding—may be biotinylated to enhance surface carbohydrate capture (Liu et al., 2022, New Journal of Chemistry, 46 (9), 4300-4306. https://doi.org/10.1039/D1NJ05758H).
(d) Biotinylated lectins (carbohydrate-binding proteins) can be immobilized on the array surface to capture specific carbohydrates (Rosenfeld et al., 2007, Journal of Biochemical and Biophysical Methods, 70 (3), 415-426. https://doi.org/https://doi.org/10.1016/j.jbbm.2006.09.008).

### 3.4 Lipid Biotinylation

Lipids can be indirectly biotinylated via an intervening lipid tether (oligo (ethylene glycol)-stearyl moieties, telechelics, or DphyTL), which also acts as a spacer between the lipid and the array surface. (Girard-Egrot OfeliaTI, 2021, Applied Sciences, Vol. 11. https://doi.org/10.3390/app 11114,876); (Sumino et al., 2011, Biomacromolecules, 12 (7), 2850-2858. https://doi.org/10.1021/bm200585y); (Lahiri, Jonas, Frutos, Kalal, & Fang, 2001, Biomedical Microdevices, 3 (2), 157-164. https://doi.org/10.1023/A: 1011406511454).

### Example 4: Printing of Biotinylated CYP450 Proteins on Streptavidin-Coated Slides

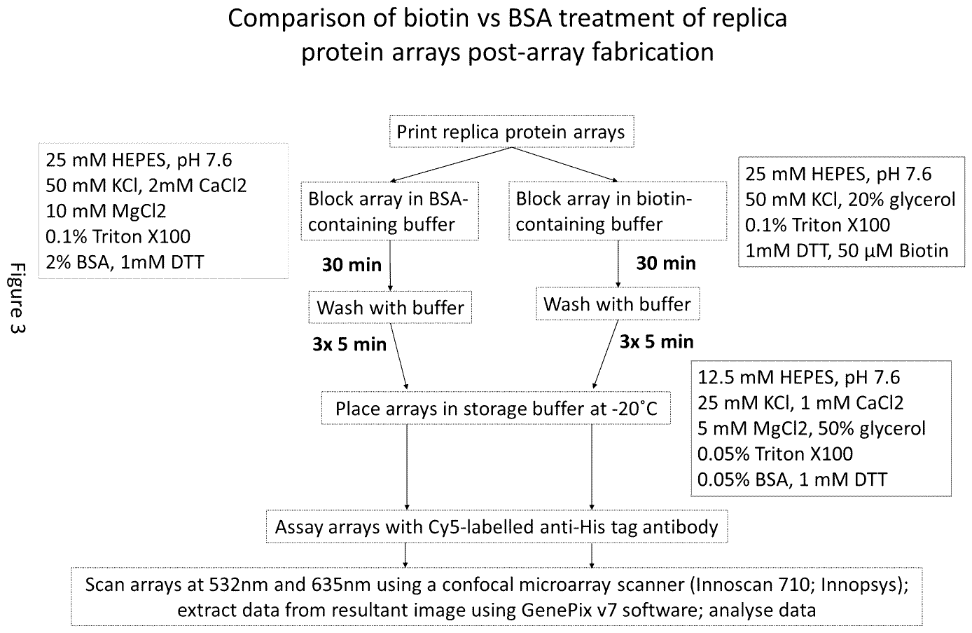

The resultant crude lysates prepared from Spodoptera frugiperda cells as described in Example 2 were printed directly on to streptavidin-coated PEG hydrogel slides as prepared in Example 1. Notably, a 50 μL aliquot of lysed insect cells expressing an individual biotinylated, BCCP-tagged recombinant protein provided enough source material to print 25 replica slides in 4-plex format, with each protein printed in triplicate in each sub array using solid pin printing methods (300 μm flat tipped pins). 3 mL of each recombinant insect cell culture thus contains enough expressed biotinylated, BCCP-tagged protein to fabricate at least 700 replica sub-arrays, or >2000 replica spots of each protein. The printed slides were washed in phosphate buffer saline (PBS) (pH 7.5; 0.2% Tween-20) to remove unbound protein, dried by centrifugation at 2000 g and kept under a gentle air stream for 5 min to ensure complete drying. Slides were fixed onto the piezoelectric stage using double sided tape and confirmed to be flat using a spirit level. An example of the spot uniformity achievable with the described method is shown in FIG. 1B, which illustrates fluorescence from Cy5 biotin BSA spots. FIG. 2 shows confirmation of binding of His-tagged CYP450 to the slide using an anti-6×His probe.

### Example 5: Use of Free Biotin to Block Unoccupied Biotin Binding Sites on the Streptavidin Tetramers

After binding of biotinylated, BCCP-tagged proteins to the streptavidin-coated surface, the slides were treated with a solution of 50 μM free biotin in 25 mM HEPES, pH 7.6, 50 mM KCl, 0.1% Triton X100, 20% glycerol, 1 mM DTT for 30 min. Without being bound by theory, it is thought that biotin binds to the streptavidin tetramer co-operatively, with each successive binding event to a sub-unit of the tetramer inducing a conformational change in the other sub-units, which increases the affinity of the remaining binding sites for binding free biotin. The present inventors have demonstrated that treatment with free biotin reduces the background, non-specific background binding to the underlying streptavidin surface by a factor of 60 when compared with blocking of slides using BSA treatment, further increasing the signal to noise ratio. A flow chart illustrating the experimental method for comparing the blocking of slides using free biotin or BSA is shown in FIG. 3.

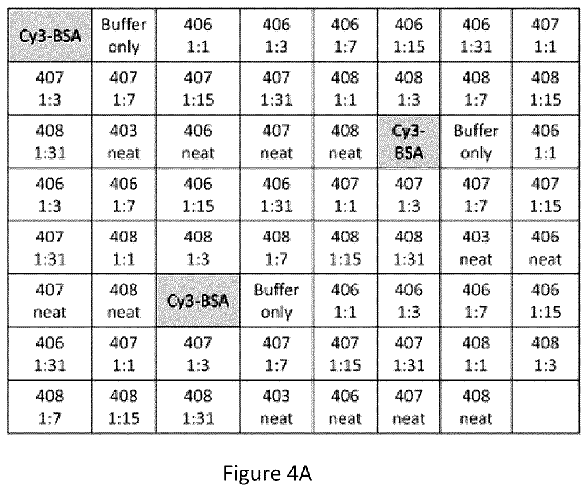

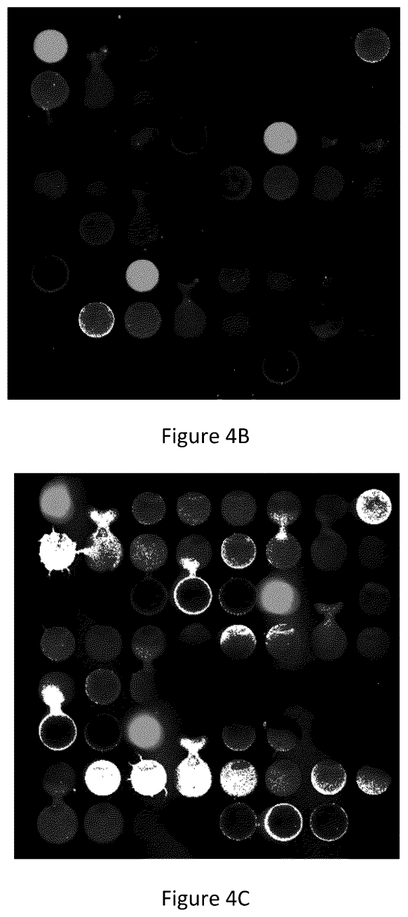

The results of a comparison of microarrays blocked using the two blocking techniques is shown in FIG. 4. The proteins on the array were BCCP-tagged full length or truncated SARS-COV-2 Spike protein or Nucleocapsid protein antigens. FIG. 4A shows the microarray layout, wherein “403” indicates the full-length SARS-CoV-2 Spike ectodomain protein-BCCP-His₆ fusion protein; “406” indicates the SARS-COV-2 Nucleocapsid protein core domains-BCCP-His6 fusion protein; “407” indicates the C-terminal domain of SARS-COV-2 Nucleocapsid protein-BCCP-His₆ fusion protein and “408” indicates the N-terminal domain of SARS-COV-2 Nucleocapsid protein-BCCP-His₆ fusion protein. FIG. 4B shows the array image, scanned at 532 and 635 nm, for the biotin-blocked array and FIG. 4C shows the array image, scanned at 532 and 635 nm, for the BSA-blocked array.

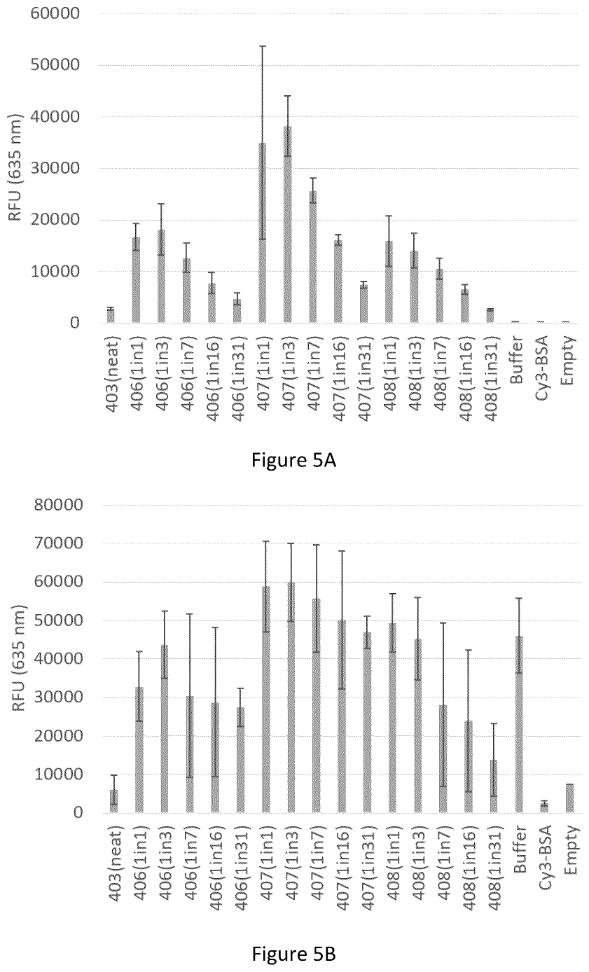

A comparison of the foreground signal intensity (at 635 nm) of the printed spots is shown in FIG. 5A (biotin-blocked array) and FIG. 5B (BSA-blocked array). N.B. The expectation is that the Foreground signal should follow a sigmoidal curve, proportional to the concentration of the antigen that was printed on to the array surface. This is clearly observed for the biotin-blocked arrays but not for the BSA-blocked arrays. N.B. the C-terminal domain of SARS-COV-2 Nucleocapsid protein-BCCP-His₆ fusion protein (“407”) printed neat had a low median RFU (<10,000) due to the aggregated signal at the edge of the spot resulting in a high signal intensity at the edge of the spot and a lower signal intensity towards the centre of the spot, resulting in a “coffee-ring” effect. However, this effect is decreased when the lysate is diluted 2-fold in lysis buffer, leading to an increase in median RFU, and further ameliorated at higher lysate dilutions resulting in the expected linear decrease in signal with increasing dilution. Slides blocked with BSA post-print also had a lower median signal for the replica spots of the neat protein (again showing the “coffee-ring” morphology), but in contrast to blocking with biotin, the signal does not decrease linearly at high lysate dilutions.

Foreground signal intensity (635 nm) coefficient of variation (CV) values for replica spots of the same antigen are shown in the following table.

| Protein | Biotin | BSA |
|---|---|---|
| 403 (neat) | 11.35085 | 63.58147 |
| 406(1in1) | 15.68956 | 27.85823 |
| 406(1in3) | 27.23227 | 20.04445 |
| 406(1in7) | 22.69955 | 70.12245 |
| 406(1in16) | 26.66794 | 67.39537 |
| 406(1in31) | 26.00837 | 18.06059 |
| 407(1in1) | 53.72981 | 20.04391 |
| 407(1in3) | 15.36942 | 16.94689 |
| 407(1in7) | 9.598893 | 25.03974 |
| 407(1in16) | 6.304609 | 35.83318 |
| 407(1in31) | 9.835669 | 8.940065 |
| 408(1in1) | 31.15524 | 15.40692 |
| 408(1in3) | 24.35913 | 23.93827 |
| 408(1in7) | 19.8847 | 76.01644 |
| 408(1in16) | 15.25184 | 77.29815 |
| 408(1in31) | 9.346031 | 68.68045 |
| Buffer | 90.62356 | 21.1657 |
| Cy3-BSA | 2.002371 | 30.01747 |
| Empty | #DIV/0! | #DIV/0! |

CVs for the Foreground signal of replica spots are in general lower for the biotin-blocked arrays than for the BSA-blocked arrays.

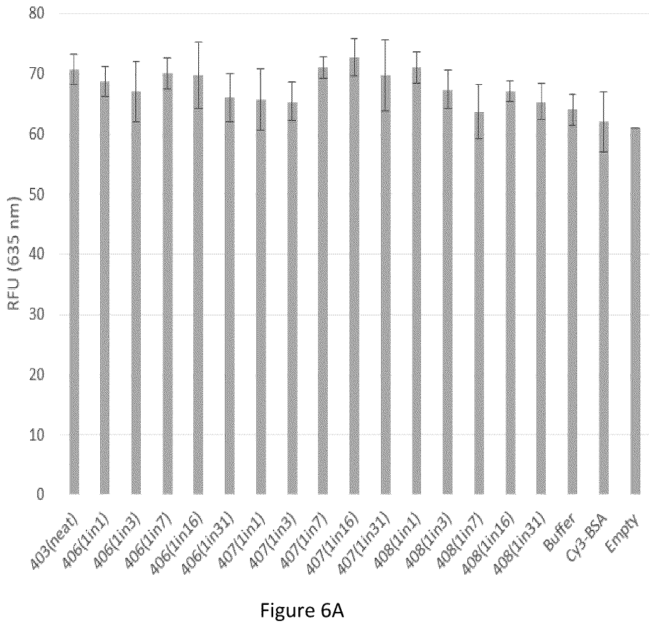

A comparison of the signal intensity (at 635 nm) of surrounding background areas adjacent to printed spots is shown in FIG. 6 for a biotin-blocked array (FIG. 6A) and a BSA-blocked array (FIG. 6B). The background signal intensity is >60-fold lower and more uniform for the biotin-blocked arrays than for the BSA-blocked arrays (note the different scales on the y axes of the graphs).

Background signal intensity (635 nm) CV values for replica spots of the same antigen are shown in the following table.

| Protein | Biotin | BSA |
|---|---|---|
| 403 (neat) | 3.561243 | 65.86434 |
| 406(1in1) | 3.664968 | 19.97227 |
| 406(1in3) | 7.462687 | 25.78731 |
| 406(1in7) | 3.779645 | 56.77699 |
| 406(1in16) | 7.905604 | 62.11323 |
| 406(1in31) | 6.060606 | 27.06629 |
| 407(1in1) | 7.664807 | 41.07873 |
| 407(1in3) | 4.92023 | 57.00129 |
| 407(1in7) | 2.439508 | 50.62399 |
| 407(1in16) | 4.204198 | 58.95314 |
| 407(1in31) | 8.410716 | 40.1221 |
| 408(1in1) | 3.72641 | 27.92745 |
| 408(1in3) | 4.774085 | 41.72256 |
| 408(1in7) | 7.082591 | 30.91657 |
| 408(1in16) | 2.58515 | 66.93198 |
| 408(1in31) | 4.676098 | 75.25421 |
| Buffer | 4.133986 | 17.08847 |
| Cy3-BSA | 8.064516 | 31.16766 |
| Empty | #DIV/0! | #DIV/0! |

CVs for the Background signal of replica spots are significantly lower for the biotin-blocked arrays than for the BSA-blocked arrays.

These data also exemplify an additional method of controlling the final density of the polypeptide analyte on the surface—by altering the concentration of the analyte in the solution that is printed, it is possible to control the amount of immobilised analyte. Unexpectedly however, the present inventors have observed that this only works as expected when done in conjunction with the biotin blocking treatment. Accordingly, without being bound by theory, it is considered that this indicates that, in the absence of the biotin blocking treatment, a proportion of analyte in each spot is non-specifically bound to the surface. The use of a biotin blocking step therefore advantageously reduces or avoids the non-specific binding of BCCP-tagged analyte to the surface of the microarray.

### Example 6: Investigation of the Surface Density of Immobilised Proteins

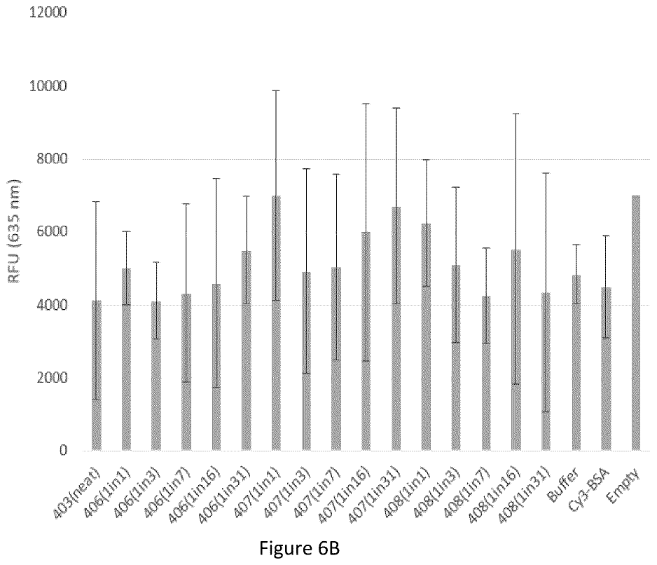

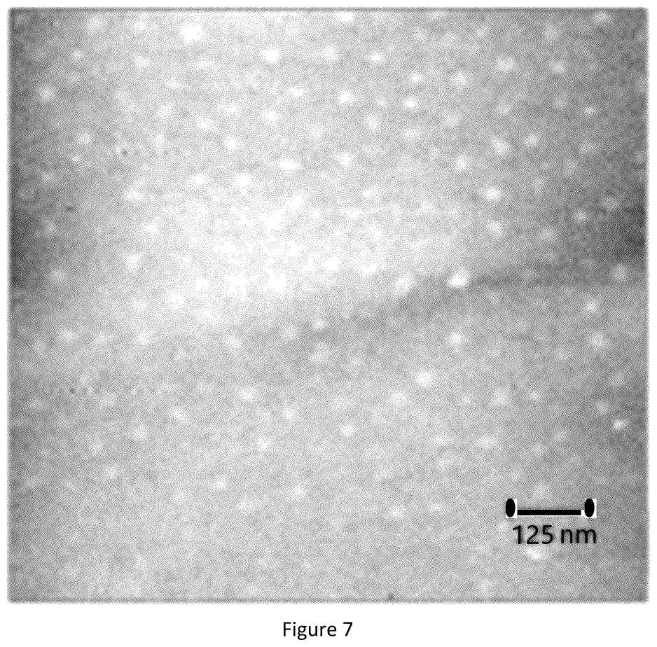

An atomic force microscope (AFM) (Easyscan 2 AFM, Nanosurf) was used in contact mode with AFM pin of thickness 2 μm (Nanosensors, Silicon SPM sensor). Areas of spots containing immobilised protein were selected at random and approached with the tip; tip scans were performed in scanning mode using either 50 μm² or 1 μm² areas. Images were analysed using the Easyscan 2 software to measure distance between immobilised recombinant proteins. Results from this analysis (see FIG. 7) revealed that the surface coverage of immobilised recombinant proteins within each spot was <10%. Further analysis using AFM showed that there is an average of 202.67+-33.31 streptavidin tetramer units per micrometre squared of surface. Accordingly, a single microarray spot with a diameter of 200 μm has approximately 6 000 000 streptavidin binding units, with an average distance of 52.87+/−3.11 nm between CYP450-bound streptavidin tetramers.

### Example 7: Comparison of a Microarray with Analyte Bound at Low Surface Density with a Microarray with Analyte Bound at High Surface Density

A low density microarray slide prepared according to the present invention is compared with an alternative microarray slide in which the analyte is bound at higher density (i.e. there is a lower intermolecular distance between the individual bound analyte molecules), such as the commercially available HuProt slide (CDI Laboratories, Inc., Puerto Rico, USA).

In particular, the microscopic features, and the signal:noise ratio performance of the two slides are compared and contrasted using the experimental methods disclosed elsewhere in the present experimental examples.

In a further experiment, a number of microarrays having a range of surface densities of bound analyte are generated. This is achieved by preparing slides having a range of different levels of NHS derivatisation of PEG moieties (e.g. 20:80, 50:50 and 100:0 derivatised:non-derivatised). The microscopic features and the signal:noise ratio performance these slides are compared as described above.

### Example 8: Investigation of the Impact of Different Conditions on the Binding of Molecules to a Microarray

In this example, the impact of varying the concentration of the linking moiety (e.g. streptavidin), varying the pH, including a competitor molecule (such as a molecule containing a free amine group) or utilising an aged slide was investigated. In particular the impact on the density of binding of the linking moiety/analyte was investigated.

### 1 METHODS & MATERIALS

1.1 Coating H Slides with Streptavidin

All microarray incubation and wash steps were performed at room temperature (RT), at 150 rotations per minute (RPM) and protected from light.

Lyophilised streptavidin (Prospec) was equilibrated at RT and resuspended in slide coating buffer (NaPO4 with 0.001% Tween-20) at pH 8.5 to 10 mg/ml. The streptavidin was further diluted to 2, 1, 0.5, 0.25, 0.125, 0.0625 and 0.03125 mg/ml in slide coating buffer or slide coating buffer with 50 mM glycine. The streptavidin was also diluted to 1 mg/ml in slide coating buffer at pH 9 or pH 4.5. Negative controls (not derivatised with streptavidin) included slide surface with slide buffer only, slide buffer with 50 mM glycine or a well without a solution. Nexterion H slides (Schott) were equilibrated at RT for 1 hour. The equilibrated H slide was assembled in a clean 24-plex gasket (GraceBio). The 90 μl of each diluted streptavidin or control was added to their respective gasket wells and incubated for 1 hour. The gasket wells were briefly rinsed three times with 200 μl wash buffer (KPO₄), the slide removed from the gasket and placed in a quadriperm dish containing 4 ml wash buffer. The slides were washed for 5 minutes; this step was repeated twice. The slide was subsequently blocked with slide coating buffer containing 50 mM glycine for 1 hour. The slides were washed three times with wash buffer, 5 minutes per wash, and finally with 4 ml water for 5 minutes before drying at 1200×g for 2 minutes at 23 °C. The dried slides were placed in slide containers, closed and sealed with parafilm and stored at −20 °C until needed.

1.2 Detecting Streptavidin on the Slide Surface with Cy3-Labelled Biotin-BSA

Slides derivatised with streptavidin under various conditions (see section 1.1) and a Nexterion HS slide (Schott) were equilibrated for 1 hour at RT and then incubated with 3 ml 40 μg/ml Cy3-labelled biotinylated BSA (Cy3-biotin-BSA) in phosphate buffered saline (PBS) pH 7.5 containing 0.2% Tween-20 (PBST) for 30 minutes. The slides were washed twice with PBST, 5 minutes per wash, and then washed twice with PBS, 5 minutes per wash. The slides were dried by centrifugation at 1200×g for 2 minutes at 23 °C and scanned with the Innoscan 710 (Innopsys, Carbonne, France) at 1% gain, 10 mW power and 10 μm pixel size.

The data was extracted using the MAPIX software (Innopsys) and subsequently processed using the i-Ome IA application to filter the data. K_{D}- and Bmax-values were obtained with GraphPad Prism (version 10.1.1)

### 1.3 Scanning Electron Microscopy

The H slides derivatised with streptavidin under various conditions (see section 1.1) and detected with Cy3-biotin-BSA were coated with a 5 nm thickness layer of carbon using a Quorum Q150VEplus combined carbon/splutter coater and then scanned by scanning electron microscopy (SEM) using the Tescan MIRA3 electron microscope under 100 000× magnification and a voltage of 10 kV.

### 1.4 Gene Synthesis and Cloning of the SARS-COV-2 Nucleocapsid (N) and Spike(s) Protein Variants

The cDNA of SARS-COV-2 Spike(S) gene, positions 1-1273, and the cDNA of SARS-COV-2 nucleocapsid (N) gene of MN908947.3 (Wuhan-hu-1, China) were chemically synthesized (GeneArt, ThermoFisher Scientific). For the recombinant expression of correctly assembled intact S protein in vivo, R682S mutation was included with the synthesized cDNA sequences to prevent the furin mediated S1/S2 cleavage within the S protein. The cDNA sequences were each cloned into a proprietary Escherichia coli/Spodoptera frugiperda transfer vector, pPRO8, such that the constructs encoded the S protein and the C-terminal domain (CTD) construct of the full-length N protein as in-frame fusions to a C-terminal Biotin Carboxyl Carrier Protein (BCCP) fused to a c-Myc followed by a hexahistidine sequence. pPRO8 is a derivative of pTriEx1.1 (Sigma, St Louis, MO, USA) and encodes the E. coli BCCP domain (amino acids 74-156 of the E. coli accB gene) downstream of a viral polyhedrin promoter and cloning sites; flanking this polh-BCCP expression cassette are the baculoviral 603 gene and the 1629 gene to enable subsequent homologous recombination of the construct into a replication-deficient baculoviral genome (Blackburn, J. M.; Shoko, A. Protein Function Microarrays for Customised Systems-Oriented Proteome Analysis. In Protein Microarrays; Korf, U., Ed.; Methods in Molecular Biology; Humana Press: Totowa, NJ, 2011; Vol. 785, pp 305-330. https://doi.org/10.1007/978-1-61779-286-1_21).

Cell pellets of 3 ml cultures of Spodoptera frugiperda Sf9 III cells were harvested and resuspended in 400 μl of Insect cell lysis buffer (25 mM Hepes, 50 mM KCL, 20% glycerol, 0.1% Triton X-100, 0.25% NaDeoxycholate, 0.5 mM TCEP, HALT protease inhibitor cocktail (EDTA free) (ThermoScientific) and 10 U of Pierce Universal Nuclease for cell lysis (ThermoScientific). Cells were lysed at room temperature for 30 mins with 100 rpm shaking. Lysates were further clarified at 15 000×g for 15 mins at 4 °C and aliquoted into fresh eppendorfs for storage until use at −80 °C.

### 1.5 Printing

Nexterion H slides derivatised with streptavidin under various conditions (see section 1.1) were printed in triplicate in a 24-plex format with the neat S protein lysate or neat C-terminal domain (CTD) construct of the N protein protein lysate. The CTD protein lysate was also printed neat, or diluted 1:1, 1:3, 1:7, 1:15 or 1:31 in insect cell lysis buffer and printed in triplicate on Nexterion HS slides in a 16-plex format. The printed slides were blocked in a biotin-containing buffer (25 mM HEPES, 50 mM KCl, 20% glycerol, 0.1% Triton X100, 1 mM DTT and 50 mM biotin) for 30 minutes, and one slide printed with CTD blocked in a BSA containing buffer (25 mM HEPES, 50 mM KCl, 20% glycerol, 0.1% Triton X100, 1 mM DTT and 0.1% BSA) was then transferred to a pap jar containing storage buffer (12.5 mM HEPES, 25 mM KCl, 1 mM CaCl2, 5 mM MgCl2, 0.05% BSA, 0.05% Triton X100 and 50% glycerol) and stored at −20 °C until needed.

1.6 Detecting Printed Proteins with Anti-c-Myc or Anti-his Antibody

Printed slides containing the neat S and CTD protein lysates or the dilution series of the CTD protein lysates were removed from storage buffer and washed three times with ice-cold PBS, 5 minutes per wash. The S and CTD protein slides were then incubated for 30 minutes with 3 ml Cy3-labelled anti-c-Myc antibody (Invitrogen) diluted 1:400 in PBST, whereas the slides containing the diluted CTD proteins were incubated for 30 minutes with 3 ml Alexa Fluor™ 647-labelled anti-His (Invitrogen) diluted 1:50 in PBST. The slides were washed twice with PBST, 5 minutes per wash and then twice with PBS, 5 minutes per wash. The slides were dried at 1200×g for 2 minutes at 23 °C and scanned using the 532 nm laser at 70% gain, 10 mW power and 10 μm pixel size. The data was extracted using the MAPIX software and filtered Ising the i-Ome AI software.

### 1.7 Patient Assays Detecting Anti-S Protein Antibodies

Printed slides containing the S protein were removed from storage buffer and washed three times with ice-cold PBS, 5 minutes per wash. Each slide was then incubated with a serum sample from a participant when they had previously tested positive for having anti-S protein antibodies (positive control) as well as with a serum sample from the same participant when they had tested negative for anti-S protein antibodies (negative control). The serum incubation was for 1 hour and the slide was subsequently washed with PBST, 5 minutes per wash. The slides were incubated with 1.25 μg/ml Alexa Fluor™ 647-labelled anti-Human IgG for 30 minutes and washed twice with PBST, 5 minutes per wash and then twice with PBS, 5 minutes per wash. The slides were dried at 1200×g for 2 minutes at 23 °C and scanned using the 635 nm laser at 10% gain, 10 mW power and 10 μm pixel size. The data was extracted using the MAPIX software and filtered using the i-Ome AI software.

### 2. RESULTS

### 2.1 Streptavidin Coating Uniformity on Nexterion H Slides

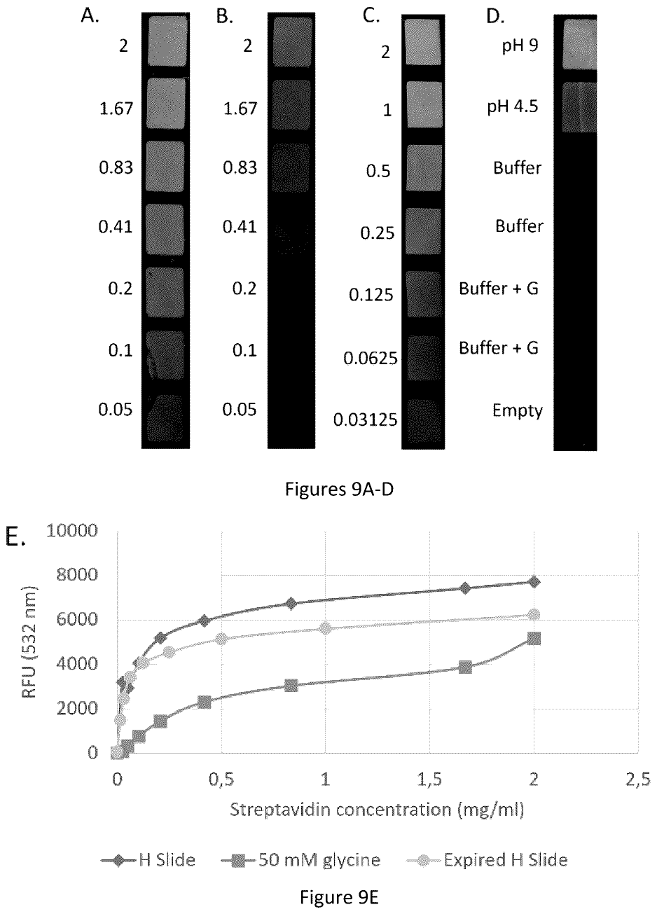

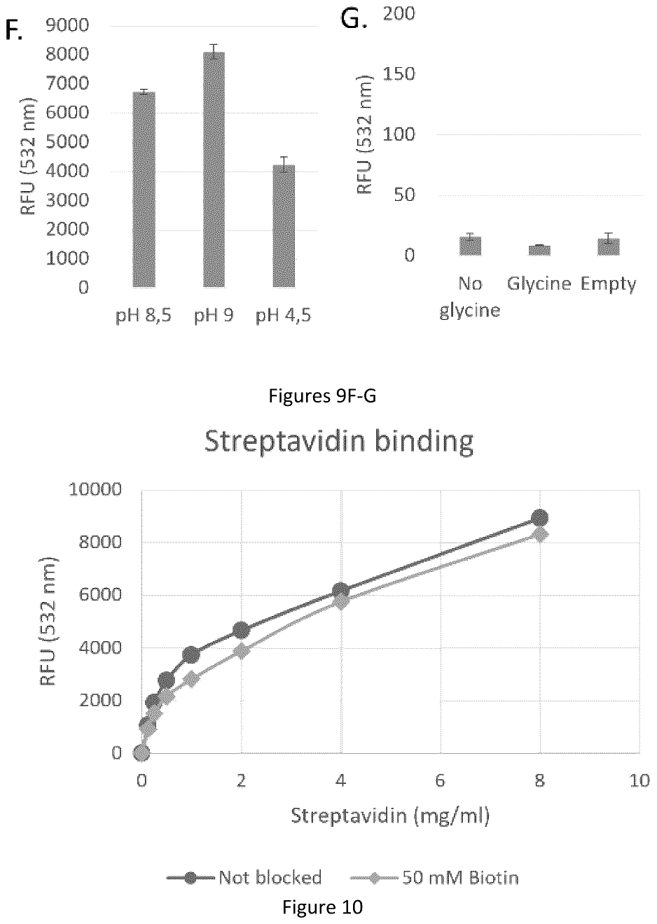

The H slides were coated with streptavidin at various concentrations alone (FIG. 9A), co-incubated with 50 mM glycine to assess competitive binding (FIG. 9B) or coated at various concentrations on an aged H slide which will have undergone spontaneous hydrolysis (FIG. 9C). Additional experiments included derivatisation with 1 mg/ml streptavidin at pH 9 or pH 4.5, as well as various controls which include slides incubated with slide coating buffer only, slide coating buffer with 50 mM glycine or an empty gasket well (FIG. 9D). The streptavidin was visualised on the slide surface using Cy3-labelled biotin-BSA as described in section 1.2.

FIG. 9E depicts the binding curves for streptavidin in the absence of or in the presence of 50 mM glycine on a fresh slide, or streptavidin in the absence of glycine on an aged slide. The K_{D} and the B_{MAX} were higher for the fresh H slides (K_{D}=0.04; B_{MAX}=5724) compared to the aged H slide (K_{D}=0.03; B_{MAX}=5509); whereas the K_{D} of the fresh H slide is lower and the B_{MAX} of the fresh H slide is higher compared to the H slide derivatised with streptavidin co-incubated with 50 mM glycine (K_{D}=0.2386; B_{MAX}=2684). Coating with 1 mg/ml streptavidin at pH 9 resulted in a higher average intensity, suggesting that more streptavidin was coated on the slide surface at pH 9, whereas slide coating with 1 mg/ml streptavidin at pH 4.5 resulted in a lower average intensity (FIG. 9F).

In a separate experiment, H slides were coated with 8, 4, 2, 1, 0.5, 0.25 or 0.125 mg/ml streptavidin, then coated with Cy3-biotin-BSA and lastly blocked with 50 mM biotin or a buffer only control (i.e., no biotin). The relative fluorescence units (RFU) reading is lower for streptavidin-coated areas which have been blocked compared to unblocked areas. For a typical binding curve, the signal saturates when all binding sites are occupied, resulting in a sigmoidal binding curve. However, non-specific binding results in a binding curve that does not plateau, but rather the signal continues to increase even though all binding sites are occupied which is due to non-specific binding. This non-specific binding is depicted for microarrays blocked with or without biotin (FIG. 10), where a second binding curve starts at 1 mg/ml streptavidin indicating that non-specific binding of streptavidin and/or Cy3-biotin-BSA to the slide surface is occurring.

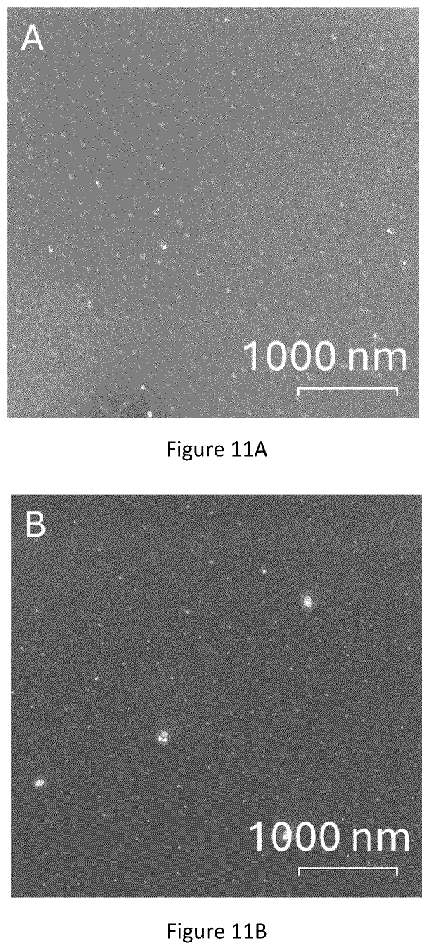

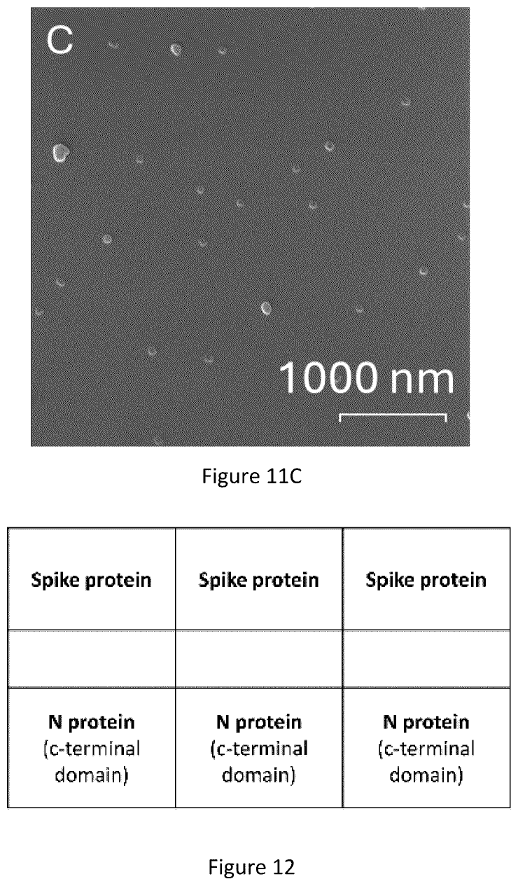

Scanning electron microscope (SEM) images of slide areas derivatised with 8 mg/ml streptavidin (FIG. 11A), 0.25 mg/ml streptavidin (FIG. 11B) and a coating buffer control (FIG. 11C) and incubated with Cy3-biotin-BSA, which is BSA that has been chemically modified with biotin and Cy3, reveal average spot diameters of 38±10 nm, 14±4 nm and 77±15 nm, respectively. The average distance between spots was 159±47 nm, 193±90 nm and 763±368 nm, respectively. The number of spots per 1 μm² for slide areas coated with 8 mg/ml streptavidin, 0.25 mg/ml streptavidin and a coating buffer control was 23, 18 and 2, respectively. N.B., the reference to “spots” in this paragraph means a streptavidin moiety, e.g. a streptavidin tetramer, bound to Cy3-biotin-BSA. The “spots” seen on the slide derivatized with coating buffer control may have been salt crystal artefacts.

### 2.2 Detecting Recombinant Proteins on Streptavidin-Coated H Slides Using Anti-c-Myc Antibody

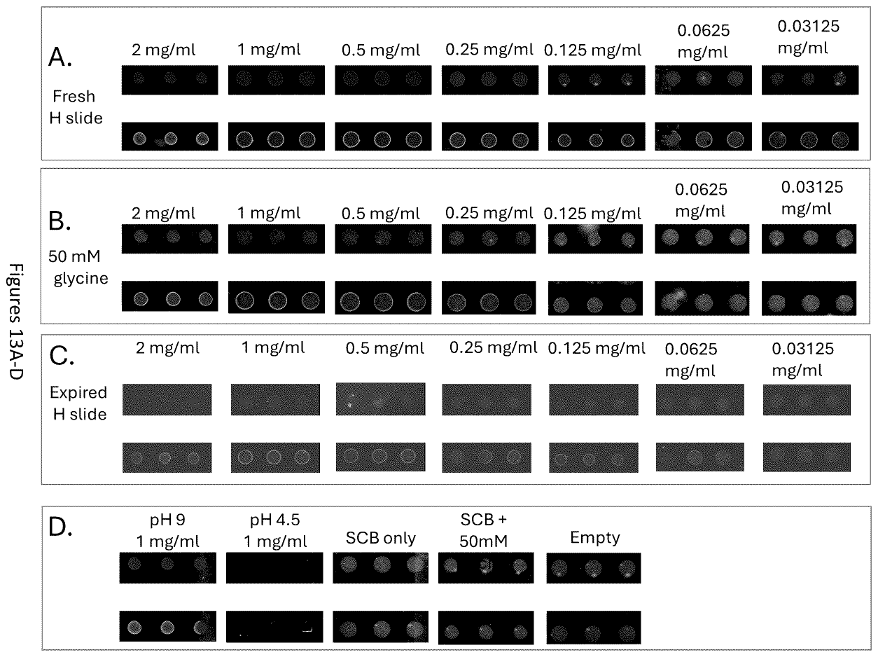

Biotin carboxyl carrier protein (BCCP)-tagged recombinant SARS-COV-2 proteins, including the full-length Spike(S) protein and the C-terminal domain (CTD) construct of the N protein, were printed in triplicate on the streptavidin-coated H slides (FIG. 12). At higher streptavidin concentrations (0.5-2 mg/ml), the S protein appears to aggregate at the edge of the spot to form a high signal intensity at the edge of the spot and a lower signal intensity towards the centre of the spot, resulting in a “coffee-ring effect”; this effect is ameliorated in areas where the streptavidin concentration is decreased to the range 0.25 mg/ml to 0.03125 mg/ml, suggesting the S protein does not aggregate at lower streptavidin concentrations (FIG. 13A). The CTD protein also forms spots with the “coffee-ring” effect. For both the glycine-treated areas (FIG. 13B) and the aged H slide (FIG. 13C), the S protein did not display the “coffee-ring” effect, however, the signal intensity appears to increase in slide areas coated with lower streptavidin concentrations. The “coffee-ring” effect for the CTD protein decreases in areas coated with 0.125-0.03215 mg/ml streptavidin (50 mM glycine). The S and CTD proteins were spotted in areas coated with streptavidin at pH 9 and pH 4.5, as well as slide areas that were not derivatised with streptavidin (FIG. 13D). For the area derivatised with streptavidin at pH 9, the “coffee-ring” effect is evident for the S protein and the CTD protein. Similar spot morphologies are evident for proteins printed in areas derivatised with streptavidin at pH 4.5, however, the signal intensity is lower. Proteins were spotted on slide surfaces not derivatised with streptavidin (FIG. 13D), including in slide coating buffer (SCB) (SCB only), in slide coating buffer containing 50 mM glycine (SCB+50 mM glycine) and without buffer (empty), suggesting that lysate proteins bound to the slide surface despite the blocking of free PEG NHS-esters with 50 mM glycine. Spots printed in the buffer control area did not have the “coffee-ring effect”, supporting the argument that the “coffee-ring” effect corresponds to the presence of streptavidin at high concentration on the slide surface.

### 2.3 Anti-Human IgG Antibody Assay

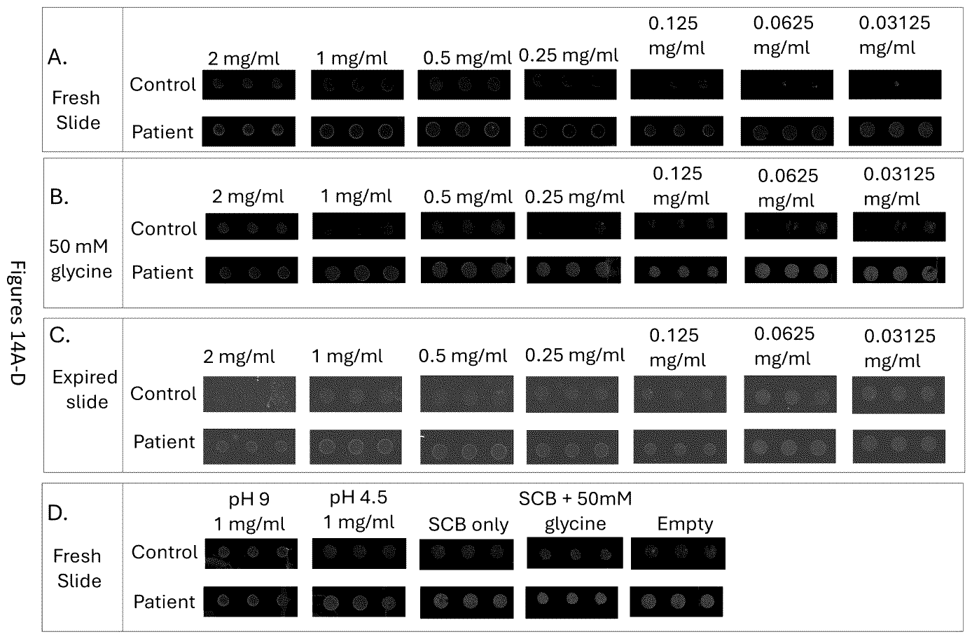

The spot morphologies for the anti-c-Myc assay were also observed for the anti-human IgG antibody assay, including the “coffee-ring” effect for the BCCP-labelled S protein printed in areas with higher streptavidin levels (FIG. 14A), which decreased in areas derivatised with streptavidin at lower concentrations, in the presence of glycine (FIG. 14B) or on the aged H slide (FIG. 14C). A high RFU was detected for patient versus control samples for slide areas derivatised at pH 9 or pH 4.5, however, the “coffee-ring effect” is still observed. Although the slide surface was blocked with glycine, proteins that discriminate patient and control samples (i.e. anti-S antibodies) were still detected on the slide surface suggesting that recombinant S proteins were spotted in glycine-blocked areas without streptavidin (FIG. 14D). Despite the “coffee-ring effect”, a stronger signal intensity is observed for S proteins exposed to patient versus control plasma for all conditions except assays on the aged H slide.

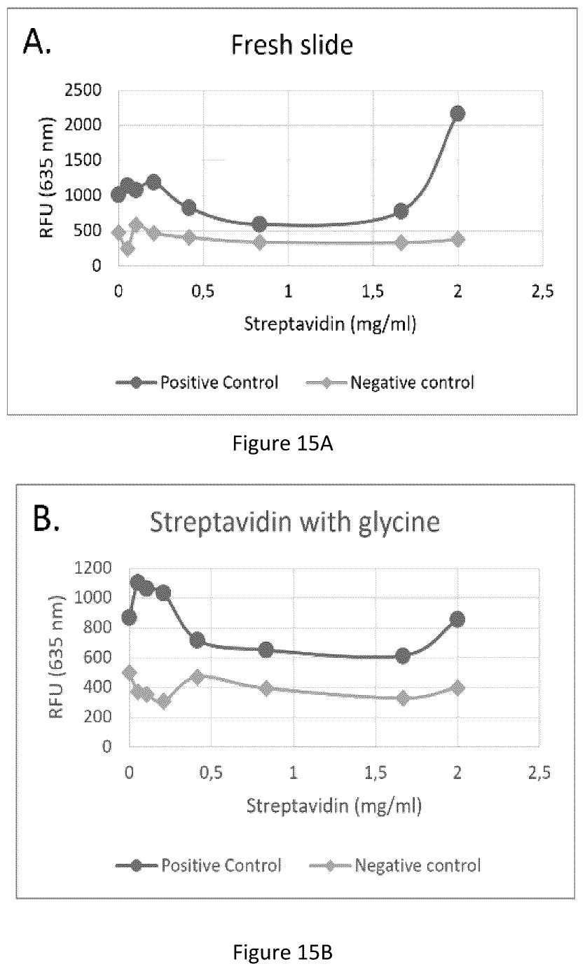

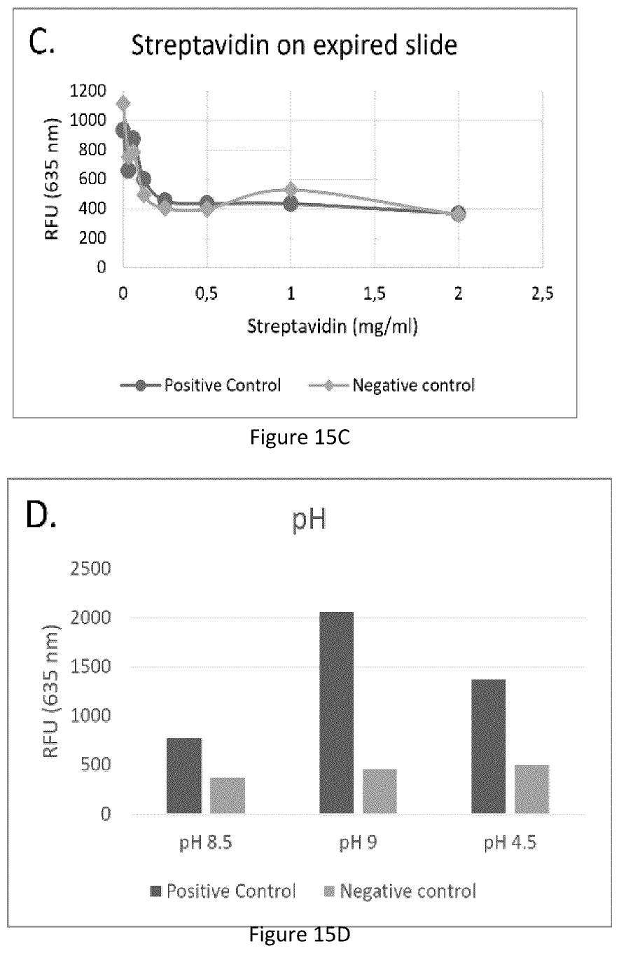

The median pixel intensity of each spot reflects the image of signal distribution of the spotted protein. A greater difference in RFU was detected between patient and control samples on areas coated with lower streptavidin concentrations, except at the 2 mg/ml streptavidin which results in the largest difference between patient and control samples (FIG. 15A), however, the higher signal likely forms because of the non-specific binding in the “coffee-ring” rather than specific antibody signal towards native S protein. Areas derivatised with streptavidin in the presence of 50 mM glycine also display a greater difference between patient and control sample at lower streptavidin concentrations (FIG. 15B), however, the binding curves are similar for patient and control samples assay on aged slides derivatised with streptavidin (FIG. 15C). The difference in RFU between patient and control samples are greater in areas derivatised with streptavidin at pH 9 and pH 4.5 (FIG. 15D), however, the “coffee-ring” spot morphology is still observed and so the increased signal intensity may still derive from non-specific binding.

### 3. DISCUSSION

The Nexterion H slide is a glass slide coated with NHS-activated polyethylene glycol (NHS-PEG) which reacts with primary amines found on lysine residues and the N-terminus of proteins. The distribution of native streptavidin on the H slide surface depends on several slide coating conditions, including but not limited to the concentration of streptavidin, the presence or absence of competitor molecules, the pH of the coating buffer during slide derivatization and the age of the H slide. Our starting conditions in this experiment were to derivatize fresh H slides with 1 mg/ml streptavidin at pH 8.5. Binding curve kinetic analysis of streptavidin produced a K_{D} of 0.04, thus H slides are expected to be fully coated at 0.08 mg/ml streptavidin, indicating that the 1 mg/ml streptavidin concentration is likely too high for slide coating. Slide coating with streptavidin in the presence of 50 mM glycine, a primary amine-containing amino acid, resulted in an increased K_{D} to 0.24 due to competitive binding of streptavidin and glycine to the PEG-NHS ester. The B_{MAX} decreased to 2684 for slides coated with streptavidin in the presence of glycine compared to 5724 for slides coated with streptavidin alone, likely resulting from the competitive binding between streptavidin and glycine, resulting in fewer free PEG-NHS ester groups available for streptavidin binding. The aged and fresh slides were stored for 2 years or 1 month at −20 °C, respectively. Thus, the aged slides will have had more time to undergo spontaneous hydrolysis, resulting in fewer free PEG-NHS ester molecules available for streptavidin binding. The resulting K_{D} of the aged slide is 0.03, indicating a similar affinity of streptavidin and PEG-NHS on the fresh slides, however, the B_{MAX} decreased slightly from 5724 to 5509 likely due to fewer available PEG-NHS esters available for derivatisation due to spontaneous hydrolysis. Coating with 1 mg/ml streptavidin at pH 9 resulted in a higher average intensity, suggesting that more streptavidin was coated on the slide surface at pH 9. Lysine has a pKₐ of 10.54 and thus pH 9 would be better than pH 8.5 for driving streptavidin derivatisation on the slide surface as lysine residues on the streptavidin would be deprotonated and would therefore more readily bind to the NHS-PEG. Coating with 1 mg/ml streptavidin at pH 4.5 resulted in a lower average intensity, indicating that fewer streptavidin molecules bound to the slide surface compared to pH 8.5 or pH 9. At a lower pH, the primary amine groups on the streptavidin are protonated, and no binding to the NHS-PEG would be expected to take place; however, signal detection at pH 4.5 suggests that not all primary amine groups were protonated. Furthermore, a lower pH will drive hydrolysis of NHS ester groups on the PEG molecule, further decreasing the extent of streptavidin derivatisation on the slide surface.

Streptavidin has a high affinity for biotin with a K_{D} of ~1×10⁻¹⁴ mol/L (Green, N. M. Avidin. In Advances in Protein Chemistry; Anfinsen, C. B., Edsall, J. T., Richards, F. M., Eds.; Academic Press, 1975; Vol. 29, pp 85-133. https://doi.org/10.1016/S0065-3233 (08) 60411-8). Biotin-bound streptavidin complex has lower non-specific binding, high thermostability, is resistant to denaturants, organic solvents, proteolytic enzymes, detergents and extreme pH. A biotin blocking step can be implemented in our assays post-printing to bind the remaining free streptavidin molecules, which in turn decreases non-specific binding from macromolecules in biological samples (e.g. serum, plasma or saliva) or the detection antibody in antibody assays. Implementing the biotin blocking step after incubation with Cy3-biotin-BSA resulted in a decreased signal (FIG. 10). The higher signal on unblocked slides may arise from the non-specific binding of Cy3-biotin-BSA or macromolecules to the streptavidin surface. However, the biotin blocking may displace non-specific interactions and in turn also decrease non-specific interactions.

As shown in Example 5, printing diluted CTD lysate on the streptavidin-coated slides followed by biotin or BSA blocking resulted in decreased signal (but increased signal:noise ratio) for biotin-blocked slides compared to BSA-blocked slides. Moreover, the linear decrease in signal at higher lysate dilutions (1:3 dilution or greater) when utilising a post-print biotin blocking step indicates that the BCCP-tagged CTD protein is binding specifically to the streptavidin under those conditions. The same effect is not seen at higher lysate dilutions when utilising a post-print BSA blocking step.

The streptavidin tetramer has a diameter of ~5 nm (Kuzuya, A., Nucleic Acids Symp. Ser. 2008, 52 (1), 681-682. https://doi.org/10.1093/nass/nrn344) and the diameter of BSA is ~7.3 nm (Ahmad, Md. W., Colloids Surf. Physicochem. Eng. Asp. 2014, 450, 67-75. https://doi.org/10.1016/j.colsurfa.2014.03.011). Each streptavidin tetramer can bind 4 biotin molecules; however, steric hindrance may allow only 2 Cy3-biotin-BSA to bind each streptavidin molecule. Thus, the expected overall size of the Cy3-biotin-BSA-bound streptavidin is predicted to be less than 19.2 nm. SEM analysis of H slides coated with 0.25 mg/ml streptavidin result in an average spot size of 14±4 nm, which falls within the size range predicted for Cy3-biotin-BSA-bound streptavidin. However, H slides derivatised with 8 mg/ml streptavidin resulted in an average spot size of 38±10 nm. An estimated 9.8×10⁹ PEG NHS ester molecules are present in the 7×7 mm gasket well, whereas the number of streptavidin tetramers at 8 mg/ml is ~9.3×10¹⁶ molecules and at 0.03 mg/ml is 3.5×10¹⁴ molecules, indicating that we are in molar-excess of streptavidin for the entire range tested on the H slide. Previous reports indicate that streptavidin 2D crystal structures can form after 1 hour at 7-8 mg/ml streptavidin which also increases the adsorption properties of streptavidin (Calvert, T. L.; Leckband, D. Two-Dimensional Protein Crystallization at Solid-Liquid Interfaces. Langmuir 1997, 13 (25), 6737-6745. https://doi.org/10.1021/la970590n), suggesting that the larger structures seen on the slide surface at 8 mg/ml may be crystalised streptavidin structures that have bound specifically and/or non-specifically to Cy3-biotin-BSA. Streptavidin forms a sticky 2D S crystalline layer at higher streptavidin concentrations (Calvert, T. L.; Leckband, D. Two-Dimensional Protein Crystallization at Solid-Liquid Interfaces. Langmuir 1997, 13 (25), 6737-6745. https://doi.org/10.1021/la970590n). The spotted lysate containing the recombinant protein evaporates most rapidly at the slide-spot interface at the spot edge, resulting in an increase in recombinant protein concentration at the spot edge and an increase in the adsorption of recombinant protein at the spot edge. The higher signal intensity observed at the spot edge may be further enhanced through the adsorption of serum proteins and/or detection antibody in antibody assays-resulting in the coffee ring effect. The distance between streptavidin spots at 8 mg/ml and 0.25 mg/ml was 159±47 and 193±90 nm, respectively. These results indicate that only a few more streptavidin molecules are bound to the slide surface at higher concentrations. Taken together, the SEM results indicate that the density of the spots and the spot size both change based on experimental conditions, and a higher streptavidin concentration appears to result in aggregated streptavidin that facilitates non-specific binding on the slide surface.

The optimal slide coating conditions should facilitate the uniform distribution of appropriately spaced recombinant proteins within a printed spot on the slide surface, which in turn contributes to an assay that is both highly sensitive and specific. In order to identify the optimal slide coating conditions, H slides were derivatised with streptavidin and printed with the BCCP-labelled SARS-COV-2 S protein to assay patient and control plasma for the detection of anti-S antibodies to determine the assay conditions which best discriminate patients from controls. A greater difference in RFU was detected between positive and negative control samples on areas coated with lower streptavidin concentrations, except at the 2 mg/ml streptavidin which results in the largest difference between positive and negative control samples, however, the higher signal likely forms because of the non-specific binding in the “coffee-ring” rather than specific antibody signal towards native S protein. Areas derivatised with streptavidin in the presence of 50 mM glycine also display a greater difference between positive and negative control sample at lower streptavidin concentrations, however, the binding curves are similar for sample-types for assay on aged slides derivatised with streptavidin. Slides on which the PEG-NHS moieties were not derivatised with streptavidin, but which were blocked with glycine do not appear to bind to Cy3-biotin-BSA (see FIG. 9D), however, lysates containing recombinant proteins bind the slide surface, suggesting that lysate proteins may be binding non-specifically to the slide surface.

In conclusion, the results in this example establish that the binding density and conformation of the linking moiety and/or the analyte on the microarray surface can be successfully modulated by varying the conditions used during coating of the microarray (including the concentration of the linking moiety in the coating buffer, the presence of a competitor molecule in the coating buffer or the pH of the slide coating buffer). The results in this example further establish that a lower concentration of linking moiety (e.g. <1 mg/ml, <0.5 mg/ml or <0.25 mg/ml) and/or the presence of a competitor molecule, and/or a change in pH can be used to avoid or reduce non-specific binding and/or unwanted aggregation of the linking moiety and/or the immobilized analyte on the surface of the microarray.

1. A microarray comprising a surface to which are bound a plurality of reactive groups, wherein an analyte is immobilised on said surface via direct binding to one or more of the reactive groups or via indirect binding via a linking moiety to one or more of the reactive groups, and wherein the surface density of said analyte within at least one discrete given area of said surface is less than about 20%.

2. A microarray comprising a surface to which are bound a plurality of reactive groups, wherein an analyte is immobilised on said surface via direct binding to one or more of the reactive groups or via indirect binding via a linking moiety to one or more of the reactive groups, and wherein the surface coverage of said analyte within at least one discrete given area of said surface is less than about 20%.

3. A microarray as claimed in claim 1, wherein the surface density of said analyte within the at least one given area of said surface is less than about 10%.

4. A microarray as claimed in claim 1 or claim 3, wherein the surface density of said analyte within the at least one given area of said surface is at least about 0.05%, preferably at least about 0.5%, more preferably at least about 1%.

5. A microarray as claimed in claim 2, wherein the surface coverage of said analyte within the at least one given area of said surface is less than about 10%.

6. A microarray as claimed in claim 2 or claim 5, wherein the surface coverage of said analyte within the at least one given area of said surface is at least about 0.05%, preferably at least about 0.5%, more preferably at least about 1%.

7. A microarray as claimed in any of claims 1 to 6, wherein the number of immobilised analyte molecules within the at least one given area is fewer than about 300 per micrometre squared.

8. A microarray as claimed in any of claims 1 to 7, wherein the number of immobilised analyte molecules within the at least one given area is fewer than about 275 per micrometre squared, fewer than about 250 per micrometre squared, fewer than about 225 per micrometre squared, fewer than about 200 per micrometre squared, fewer than about 175 per micrometre squared, fewer than about 150 per micrometre squared, fewer than about 125 per micrometre squared, fewer than about 100 per micrometre squared, fewer than about 75 per micrometre squared, fewer than about 50 per micrometre squared, fewer than about 25 per micrometre squared, fewer than about 20 per micrometre squared, fewer than about 15 per micrometre squared, fewer than about 10 per micrometre squared or fewer than about 5 per micrometre squared.

9. A microarray comprising a surface to which are bound a plurality of reactive groups, wherein an analyte is immobilised on said surface via direct binding to one or more of the reactive groups or via indirect binding via a linking moiety to one or more of the reactive groups, and wherein the number density of immobilised analyte molecules within at least one discrete given area of said surface is fewer than about 300 per micrometre squared.

10. A microarray as claimed in claim 9, wherein the number of immobilised analyte molecules within the at least one given area is fewer than about 275 per micrometre squared, fewer than about 250 per micrometre squared, fewer than about 225 per micrometre squared, fewer than about 200 per micrometre squared, fewer than about 175 per micrometre squared, fewer than about 150 per micrometre squared, fewer than about 125 per micrometre squared, fewer than about 100 per micrometre squared, fewer than about 75 per micrometre squared, fewer than about 50 per micrometre squared fewer than about 25 per micrometre squared, fewer than about 20 per micrometre squared, fewer than about 15 per micrometre squared, fewer than about 10 per micrometre squared or fewer than about 5 per micrometre squared.

11. A microarray as claimed in any preceding claim, wherein said analyte is immobilised on said surface via indirect binding to one or more of the reactive groups via a linking moiety.

12. A microarray as claimed in any preceding claim, wherein said analyte comprises a tag which permits immobilisation of said analyte on said surface.

13. A microarray as claimed in any preceding claim, wherein said analyte comprises one or more of a polypeptide, a nucleic acid, a lipid and a carbohydrate.

14. A microarray as claimed in any preceding claim, wherein said analyte comprises one or more of a polypeptide and a nucleic acid.

15. A microarray as claimed in any preceding claim, wherein said analyte is a polypeptide, for example a glycoprotein.

16. A microarray as claimed in any preceding claim, wherein said analyte is a polypeptide.

17. A microarray as claimed in any one of claims 12 to 16, wherein said analyte is a polypeptide, and said tag which permits immobilisation of said polypeptide on said surface is fused to the N- or the C-terminus of the polypeptide.

18. A microarray as claimed in any one of claims 13 to 17, wherein said polypeptide is correctly folded.

19. A microarray as claimed in any preceding claim, wherein said analyte is biotinylated.

20. A microarray as claimed in claim 19, wherein said biotinylated analyte is chemically biotinylated or enzymatically biotinylated.

21. A microarray as claimed in any one of claims 12 to 20, wherein said tag which permits immobilisation of said analyte on said surface comprises a biotin carboxyl carrier protein (BCCP) motif or an Avi-tag (SEQ ID NO: 1).

22. A microarray as claimed in claim 21, wherein said BCCP motif has at least 80% sequence identity, preferably at least 90% sequence identity, more preferably 100% sequence identity to SEQ ID NO. 2.

23. A microarray as claimed in claim 21 or claim 22, wherein said BCCP motif is correctly folded.

24. A microarray as claimed in any one of claims 21 to 23, wherein biotin is bound to: (i) a biotin attachment domain within said BCCP motif or (ii) said Avi-tag.

25. A microarray as claimed in claim 24, wherein biotin is attached to said biotin attachment domain or said Avi-tag via enzymatic biotinylation, preferably via biotin ligase.

26. A microarray as claimed in any preceding claim, wherein said linking moiety is a biotin-binding molecule and said analyte is biotinylated.

27. A microarray as claimed in claim 26, wherein said biotin-binding molecule is a protein.

28. A microarray as claimed in claim 26 or claim 27, wherein said biotin-binding molecule is selected from the group consisting of (i) an anti-biotin antibody; (ii) avidin; (iii) neutravidin; (iv) streptavidin, or (iii) a fragment, variant or analogue of streptavidin, avidin, neutravidin or said anti-biotin antibody which retains biotin-binding capability.

29. A microarray as claimed in any one of claims 26 to 28, wherein said biotin-binding molecule comprises a sequence having at least 80% sequence identity, preferably at least 90% sequence identity, more preferably 100% sequence identity to SEQ ID NO. 3.

30. A microarray as claimed in any of claims 26 to 29, wherein said biotin-binding molecule is in homotetrameric form and is not aggregated.

31. A microarray as claimed in any one of claims 26 to 30 wherein said microarray comprises multiple binding sites for biotin via said biotin-binding molecules, and wherein a portion of said biotin binding sites are not bound to biotinylated analyte, and wherein said biotin binding sites that are not bound to biotinylated analyte are bound to free biotin.

32. A microarray as claimed in claim 31, wherein substantially all of said biotin binding sites that are not bound to biotinylated analyte are occupied by free biotin, for example at least 70%, at least 80%, at least 90%, at least 95%, at least 98% or at least 99% of said biotin binding sites that are not bound to analyte are bound to free biotin.

33. A microarray comprising a surface to which are bound a plurality of reactive groups, wherein a linking moiety is immobilised on said surface via binding to one or more of the reactive groups, and wherein the surface coverage of said linking moiety within at least one discrete given area of said surface is less than about 20%.

34. A microarray comprising a surface to which are bound a plurality of reactive groups, wherein a linking moiety is immobilised on said surface via binding to one or more of the reactive groups, and wherein the number of said linking moieties immobilised within at least one discrete given area of said surface is fewer than about 300 per micrometre squared, for example fewer than about 275 per micrometre squared, fewer than about 250 per micrometre squared, fewer than about 225 per micrometre squared, fewer than about 200 per micrometre squared, fewer than about 175 per micrometre squared, fewer than about 150 per micrometre squared, fewer than about 125 per micrometre squared, fewer than about 100 per micrometre squared, fewer than about 75 per micrometre squared, fewer than about 50 per micrometre squared fewer than about 25 per micrometre squared, fewer than about 20 per micrometre squared, fewer than about 15 per micrometre squared, fewer than about 10 per micrometre squared or fewer than about 5 linking moieties per micrometre squared.

35. A microarray as claimed in claim 33 or 34, wherein said linking moiety is a biotin-binding molecule.

36. A microarray as claimed in claim 35, wherein the biotin-binding molecule is as defined in any one of claims 27 to 30.

37. A microarray as claimed in any preceding claim, wherein said surface density or said surface coverage is determined by microscopy.

38. A microarray as claimed in claim 37, wherein said microscopy is selected from the group consisting of atomic force microscopy (AFM), electron microscopy and super-resolution microscopy.

39. A microarray as claimed in any preceding claim wherein said reactive groups on said surface are selected from carboxylic acid groups, activated carboxylic acid groups, amine groups, imidoester groups, maleimide groups, haloacetyl groups, pyridyl dithiol groups, azide groups, hydrazide groups, alkoxyamine groups, thiol groups, aryl azide groups and diazirine groups.

40. A microarray as claimed in any preceding claim wherein said reactive groups on said surface are selected from carboxylic acid groups, activated carboxylic acid groups, amine groups and maleimide groups.

41. A microarray as claimed in any preceding claim wherein said reactive groups on said surface are activated carboxylic acid groups, for example 1-ethyl-3-(3-dimethylaminopropyl) carbodiimide (EDC) activated or N-hydroxysuccinimide (NHS) activated carboxylic acid groups.

42. A microarray as claimed in any preceding claim wherein said reactive groups are bound to said surface via a hydrophilic organic polymer.

43. A microarray as claimed in claim 42, wherein said hydrophilic organic polymer is selected from the group consisting of polyacrylamide, polyurethane, polyethyleneimine and polyethylene glycol, preferably polyethylene glycol.

44. A microarray as claimed in claim 43, wherein said polyethylene glycol has an average molecular weight in the range 500 to 20000.

45. A microarray as claimed in any preceding claim, wherein the immobilised analytes, or the linking moieties, or the linking moieties bound to immobilised analyte on said surface are spaced at least 50 nm apart, for example at least 100 nm or at least 150 nm apart from each other, as measured by AFM.

46. A microarray as claimed in any preceding claim, wherein the at least one given area has an area of less than 0.2 mm², for example between 0.8 μm² and 0.2 mm².

47. A microarray as claimed in any preceding claim, which has multiple discrete given areas on said surface, e.g. multiple analyte spots.

48. A method of manufacturing a microarray comprising the steps of: (i) providing a surface to which are bound a plurality of reactive groups, wherein only approximately 20% or less of said reactive groups are able to react at any one time; (ii) optionally contacting said reactive groups with a linking moiety under conditions whereby said linking moiety reacts with one or more reactive groups which is able to react, resulting in the binding of the linking moiety to said surface; and (iii) depositing a sample of an analyte on the surface within at least one discrete given area of the surface such that the analyte is immobilised on said surface via direct binding to one or more reactive groups which is able to react, or via indirect binding via said linking moiety to a reactive group; wherein the surface coverage of the bound analyte within the at least one discrete given area of said surface is accordingly less than about 20%.

49. A method of reducing the density of analyte bound to the surface of a microarray comprising the steps of (i) providing a surface to which are bound a plurality of reactive groups; (ii) causing or allowing a portion of said reactive groups to be deactivated or inaccessible such that approximately 20% or less of said reactive groups are able to react at any one time; (iii) optionally contacting said reactive groups with a linking moiety under conditions whereby reactive one or more reactive groups retaining reactivity are able to react with the linking moiety resulting in the binding of the linking moiety to said surface; and (iv) depositing a sample of an analyte on the surface within at least one discrete given area of the surface such that the analyte is immobilised on said surface via direct binding to one or more reactive groups, or via indirect binding via said linking moiety to one or more reactive groups; wherein the surface coverage of the bound analyte within the at least one discrete given area of said surface is accordingly less than about 20%.

50. A method of:
i) increasing the analyte signal to background noise ratio of a microarray;
ii) increasing the rotational and conformational freedom of an analyte immobilised on a microarray; and/or
iii) increasing the proportion of physiologically-relevant interactions between analytes immobilised on a microarray and test molecules applied to said microarray;
wherein said method comprises the step of providing a microarray comprising a surface to which are bound a plurality of reactive groups, wherein the analyte is immobilised on said surface via direct binding to one or more of the reactive groups or via indirect binding via a linking moiety to one or more of the reactive groups, and wherein the surface coverage of the analyte immobilised within at least one discrete given area of said surface is less than about 20%.

51. A method as claimed in any one of claims 48 to 50, wherein said microarray is further as defined in any one of claims 1 to 47.

52. Use of a surface of a microarray as a low density surface on which the surface density of analyte immobilised within at least one discrete given area of said surface is less than about 20%, wherein the analyte is optionally immobilised on said microarray via a linking moiety.

53. Use of a surface for the manufacture of a low density microarray comprising immobilised analyte, wherein the surface density of analyte immobilised within at least one discrete given area of said surface is less than about 20% and wherein the analyte is optionally immobilised on said microarray via a linking moiety.

54. Use of reactive groups of a surface suitable for forming a low density microarray for reducing the density of analyte immobilised on said surface, wherein a proportion of said reactive groups are unable to react with said analyte such that the surface density of analyte immobilised on at least one discrete given area of said surface is less than about 20%, and wherein said analyte is optionally immobilised on said surface via linking moiety.

55. Use of reactive groups of a surface suitable for forming a low density microarray for the manufacture of a low density protein microarray, wherein a proportion of said reactive groups on a said surface are unable to react with an analyte such that the surface density of analyte immobilised on at least one discrete given area of said surface is less than about 20%, and wherein said analyte is optionally immobilised on said surface via a linking moiety.

56. Use of a microarray as claimed in any one of claims 1 to 47 for:
i) the identification of interactions between said analyte and test molecules applied to said analyte;
ii) the determination of an antibody profile of a subject;
iii) identifying a biomolecule which specifically binds to said immobilised analyte;
iv) the identification of an antibody which specifically binds said immobilised analyte and which is suitable for the diagnosis or treatment of a disease; or
v) identifying a biomolecule which specifically binds said immobilised analyte and which is capable of treating a disease mediated by said immobilised analyte.

57. The use as claimed in any one of claims 52 to 56, wherein said low density microarray is as defined in any one of claims 1 to 47.

58. A method of manufacturing a microarray comprising the steps of: (i) providing a surface to which are bound a plurality of reactive groups; (ii) contacting said reactive groups with a linking moiety comprising a biotin-binding molecule under conditions whereby said linking moiety reacts with one or more reactive groups which is able to react, resulting in the binding of the linking moiety to said surface; (iii) depositing a sample of a biotinylated analyte on the surface such that the analyte is immobilised on said surface via indirect binding via said linking moiety to a reactive group; and thereafter (iv) applying a solution of biotin to the surface of the microarray.

59. A method of manufacturing a microarray comprising the steps of: (i) providing a surface to which are bound a plurality of reactive groups; (ii) contacting said reactive groups with a solution of a linking moiety wherein the concentration of the linking moiety in said solution is less than 1 mg/ml and whereby said linking moiety reacts with one or more reactive groups which is able to react, resulting in the binding of the linking moiety to said surface.

60. A method as claimed in claim 59, wherein said method comprises a further step (iii) of depositing a sample of an analyte on the surface such that the analyte is immobilised on said surface via indirect binding via said linking moiety to the one or more reactive groups.

61. A method as claimed in claim 59 or claim 60, wherein the concentration of said linking moiety in said solution is 0.8 mg/ml or less, 0.6 mg/ml or less, 0.5 mg/ml or less, 0.4 mg/ml or less, 0.3 mg/ml or less, 0.25 mg/ml or less, 0.15 mg/ml or less, 0.1 mg/ml or less, 0.05 mg/ml or less or 0.01 mg/ml or less.

62. A method as claimed in any of claims 59 to 61, wherein the linking moiety is a biotin-binding molecule.

63. A method as claimed in any of claims 58 to 62, wherein the linking moiety is as defined in any of claims 26 to 30.

64. A method as claimed in any of claims 58 to 63, wherein the analyte is as defined in any of claims 12 to 20.

65. A method as claimed in any of claims 58 to 64, wherein the analyte is capable of binding to the linking moiety with a K_{D} of less than about 1×10⁻¹² mol/L, for example less than about 1×10⁻¹³ mol/L, for example about 1×10⁻¹⁴ mol/L.

66. A method as claimed in any of claims 58 to 65, wherein said analyte is biotinylated, for example chemically biotinylated or enzymatically biotinylated.

67. A method as claimed in any of claims 58 to 66, wherein said analyte comprises a biotin carboxyl carrier protein (BCCP) motif or an Avi-tag (SEQ ID NO: 1).

68. A method as claimed in claim 67, wherein said BCCP motif has at least 80% sequence identity, at least 90% sequence identity or 100% sequence identity to SEQ ID NO: 2.

69. A method as claimed in claim 67 or claim 68, wherein said BCCP motif is correctly folded.

70. A method as claimed in any of claims 67 to 69, wherein biotin is bound to: (i) a biotin attachment domain within said BCCP motif or (ii) said Avi-tag.

71. A method as claimed in claim 70, wherein biotin is attached to said biotin attachment domain or said Avi-tag via enzymatic biotinylation, for example via biotin ligase.

72. A method as claimed in any of claims 60 to 71, wherein said method comprises a further step (iv) of applying a solution of a blocking agent to the surface of the microarray.

73. A method as claimed in claim 72, wherein said blocking agent is selected from the group consisting of skimmed milk powder, bovine serum albumin (BSA), casein, polyethylene glycol (PEG), polyvinylpyrrolidone (PVP), gelatin, serum, and biotin.

74. A method as claimed in claim 72 or claim 73, wherein said blocking agent is able to bind to the linking moiety with a K_{D} of less than about 1×10⁻¹² mol/L, for example less than about 1×10⁻¹³ mol/L, for example about 1×10⁻¹⁴ mol/L.

75. A method as claimed in any of claims 72 to 74, wherein said blocking agent is biotin.

76. A method as claimed in any of claims 72 to 75, wherein said linking moiety is as defined in any of claims 26 to 30.

77. A method as claimed in any of claims 58 to 76, wherein said reactive groups are as defined in any of claims 39 to 44.

78. A method as claimed in any of claims 58 to 77, wherein said solution of a linking moiety further comprises a competitor molecule which is able to compete with the linking moiety for reaction with the reactive groups.

79. A method as claimed in claim 78, wherein said competitor molecule comprises a free amine group.

80. A method as claimed in claim 78 or claim 79, wherein said competitor molecule is selected from the group consisting of an amino acid and an alkanolamine.

81. A method as claimed in any of claims 78 to 80, wherein said competitor molecule is selected from the group consisting of glycine, alanine, serine, lysine, arginine, histidine, valine, isoleucine, leucine, phenylalanine, tyrosine, tryptophan, methanolamine and ethanolamine.

82. A method as claimed in any of claims 78 to 81, wherein said competitor molecule is selected from the group consisting of glycine and ethanolamine.

83. A method as claimed in any of claims 58 to 82, wherein the pH of the solution of the linking moiety is about 7.5 to about 11, for example about 8 to about 9.5 or about 8.5 to about 9, for example about 8.5.

84. A method as claimed in any of claims 58 to 82, wherein the pH of the solution of the linking moiety is about 4 to about 7.5, for example about 4.5 to 6, for example about 4.5.

85. A method as claimed in any of claims 58 and 60 to 84, wherein the sample of the analyte is deposited on the surface in the form of a solution.

86. A method as claimed in claim 85, wherein the solution of analyte is diluted before deposition on the surface, for example at least a 1 in 2 dilution, at least a 1 in 3 dilution, at least a 1 in 4 dilution, at least a 1 in 5 dilution, at least a 1 in 10 dilution, at least a 1 in 20 dilution, at least a 1 in 25 dilution, at least a 1 in 50 dilution or at least a 1 in 100 dilution.

87. A method as claimed in any of claims 58 to 86 which results in a density of said linking moiety within at least one discrete given area of said surface of fewer than about 300 linking moieties per micrometre squared, for example fewer than about 275 per micrometre squared, fewer than about 250 per micrometre squared, fewer than about 225 per micrometre squared, fewer than about 200 per micrometre squared, fewer than about 175 per micrometre squared, fewer than about 150 per micrometre squared, fewer than about 125 per micrometre squared, fewer than about 100 per micrometre squared, fewer than about 75 per micrometre squared, fewer than about 50 per micrometre squared fewer than about 25 per micrometre squared, fewer than about 20 per micrometre squared, fewer than about 15 per micrometre squared, fewer than about 10 per micrometre squared or fewer than about 5 linking moieties per micrometre squared.

88. A method as claimed in any of claims 58 and 60 to 87 which results in a density of said analyte within at least one discrete given area of said surface of fewer than about 300 analyte molecules per micrometre squared, for example fewer than about 275 per micrometre squared, fewer than about 250 per micrometre squared, fewer than about 225 per micrometre squared, fewer than about 200 per micrometre squared, fewer than about 175 per micrometre squared, fewer than about 150 per micrometre squared, fewer than about 125 per micrometre squared, fewer than about 100 per micrometre squared, fewer than about 75 per micrometre squared, fewer than about 50 per micrometre squared fewer than about 25 per micrometre squared, fewer than about 20 per micrometre squared, fewer than about 15 per micrometre squared, fewer than about 10 per micrometre squared or fewer than about 5 analyte molecules per micrometre squared.

89. A microarray obtainable by a method as claimed in any of claims 58 to 88.


SEQUENCE LISTING (4 sequences)

SEQ ID NO: 1 - protein, 15 aa, synthetic construct
GLNDIFEAQKIEWHE

SEQ ID NO: 2 - protein, 83 aa, Escherichia coli
AAAEISGHIVRSPMVGTFYRTPSPDAKAFIEVGQKVNVGDTLCIVEAMKMMNQIEADKSGTVKAILVESGQPVEFDEPLVVIE

SEQ ID NO: 3 - protein, 123 aa, Streptomyces avidinii
AEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKP

SEQ ID NO: 4 - protein, 180 aa, Streptomyces avidinii
MRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDA